(19) United States
(12) Patent Application Publication
(10) Pub. No.: US 2019/0265319 A1
(43) Pub. Date: Aug. 29, 2019

(54) SYSTEM AND METHOD FOR SMALL MOLECULE ACCURATE RECOGNITION TECHNOLOGY ("SMART")

(71) Applicant: THE REGENTS OF THE UNIVERSITY OF CALIFORNIA, OAKLAND, CA (US)

(72) Inventors: Chen Zhang, La Jolla, CA (US); Yerlan Idelbayev, La Jolla, CA (US); Garrison W. Cottrell, La Jolla, CA (US); William H. Gerwick, La Jolla, CA (US); Preston B. Landon, La Jolla, CA (US)

(21) Appl. No.: 16/319,544

(22) PCT Filed: Jul. 24, 2017
(86) PCT No.: PCT/US2017/043502

§ 371 Date: Jan. 22, 2019

Related U.S. Application Data
(60) Provisional application No. 62/365,548, filed on Jul. 22, 2016.

(51) Int. Cl. G01R 33/465; G06F 17/14; G16B 40/00

(52) U.S. Cl. CPC G01R 33/465; G16B 40/00; G06F 17/14

(57) ABSTRACT

Systems and methods are provided that leverage the advantages of Non-Uniform Sampling (NUS) 2D NMR techniques and Deep Convolutional Neural Networks (DCNN) to create the “SMART” tool that can assist in high-throughput natural product discovery. The methodological development of SMART is accomplished in two steps: (1) the NUS heteronuclear single quantum coherence (HSQC) NMR program was adapted to a state-of-the-art nuclear magnetic resonance (NMR) instrument equipped with a cryoprobe, and the data reconstruction methods were optimized, (2) a DCNN with modified contrastive loss was trained on a database containing over 2000 HSQC spectra as the initial training set. To demonstrate the utility of SMART, several newly isolated compounds were automatically located with their known analogues in the test embedding map (TEM), thereby streamlining the discovery pipeline for new biologically active natural products.

### CROSS-REFERENCE TO RELATED APPLICATIONS

This application claims benefit of priority of U.S. Provisional Patent Application Ser. No. 62/365,548, filed Jul. 22, 2016, owned by the assignee of the present application and herein incorporated by reference in its entirety.

### STATEMENT OF GOVERNMENT INTEREST

This invention was made with government support under SMA 1041755 awarded by the National Science Foundation (NSF). The government has certain rights in the invention.

### FIELD

The invention relates to the field of techniques for drug discovery.

### BACKGROUND

Approximately 70% of all approved drugs are natural products (NPs), their analogues, or a chemical modification of an existing NP. In most natural products research (NPR), traditional compound dereplication practices have entailed the collection and analysis of nuclear magnetic resonance (NMR) spectra, including running 1D and 2D NMR spectroscopic experiments for the purposes of molecular framework construction, assembly, and relative stereochemistry determination. However, 1D NMR spectra is less informative and less discriminative than 2D NMR, but as will be described 2D NMR has its own disadvantages.

More recently, mass spectrometric approaches and mass spectrometry (MS)-based molecular networking, in part stimulated by integration with DNA sequencing and genome mining, have been integrated into NPR workflows. Nevertheless, conventional NMR practices are still indispensable to the characterization and dereplication of NPs. Unfortunately, 2D NMR experiments can be time consuming, especially when the sample is relatively scarce. Furthermore, 2D NMR-based structural assignments can sometimes take a long period of time to accomplish due to the inherent structural complexity of some NPs.

Along with mass spectrometry, circular dichroism and infrared spectroscopy techniques that have developed over the past few years, state-of-the-art cryoprobe NMR instruments have pushed down the sample requirements for NPs discovery to only a few nanomoles. But the conventional stepwise unselective 2D NMR pulse sequences that are typically used require relatively large numbers of scans before Fourier Transformation of the data. Specifically, conventional 2D NMR spectroscopy applies discrete Fourier transformations such that the experiments are very time consuming, especially when the sample is scarce, and especially when generating high frequency resolution in the indirect dimension. Non-Fourier transform methods in combination with non-uniform sampling (NUS) allows high resolution along the indirect dimensions while reducing the sampling time. The NUS method is designed to reduce the number of data collection experiments while delivering an accurate estimation of the fully sampled spectrum.

To streamline compound dereplication or even structure determination, algorithms have been applied for 2D NMR spectra comparisons, such as the 2D NMR peak alignment algorithm. Invariably, these techniques are not powerful enough to accurately file 2D NMR spectra into the correct NPs family. This arises from several reasons, such as compound concentration, solvent effect, and the interactive effect of a single functional group alteration on the ¹H and ¹³C NMR chemical shifts, which all raise the difficulty for computer assisted 2D NMR data analysis. At the same time, artifacts are often introduced into NMR spectra, and this makes it extremely difficult for the existing pattern recognition or overlap methods to distinguish genuine peaks from artifacts.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

Systems and methods according to present principles meet the needs of the above in several ways, including by integrating fast 2D NMR techniques, e.g., nonuniform sampling, and so on, with deep learning such as by neural networks, e.g., deep convolutional neural networks, to enable rapid dereplication of known compounds, which then enables the connection and/or association of unknown compounds in libraries or mixed samples, e.g., such as Gerwick fractions/extracts of collected marine natural products. In more detail, a modified AI algorithm has been trained to generate an AI clustering map with a training data set. The algorithm produces a clustering of the input data set, which is based on HSQC data, based on structural similarities. In other words, similar molecules are mapped to nearby locations in the AI clustering map. The results are visualized in embedding maps with nodes representing each spectra and node distance for structural similarity. In certain implementations described here, the AI clustering map has node colors that represent compounds described in the same articles, e.g., similar compounds are indicated by being displayed using similar or the same colors.

In contrast to present systems and methods, prior art techniques may suffer from, in some cases, various deficiencies. For example, conventional 2D NMR spectroscopy cannot generate high quality high resolution 2D NMR spectra from limited amounts of compound in a short time. The conventional stepwise fashion unselective 2D NMR pulse sequence requires sampling all increments in the indirect dimension. The conventional 2D NMR spectroscopy applies discrete Fourier transform so that the experiments are very time consuming when generating high frequency resolution in the indirect dimension of the spectra. Existing non-uniform data reconstruction methods (Poisson Gap, Maximum Entropy Method, etc) alone do not generate 2D NMR spectra with a sufficient signal to noise ratio. Existing 2D NMR peak alignment algorithms are not powerful enough to accurately file 2D NMR spectra into an order of MNP families, due to compound concentration variation, solvent effects, and the gestalt effect of single functional group alternation on environmental NMR chemical shifts. Artifacts are very prone to being introduced to NMR spectra, for which the existing pattern recognition or overlap methods cannot distinguish from genuine peaks. Existing pattern recognition or overlap methods are not user friendly and do not learn new spectra by themselves.

Systems and methods according to present principles address and overcome one or more of these or other problems, and are particularly directed to a method to fast and accurately dereplicate or profile chemical compounds into compound families.

In one aspect, the invention is directed towards a method of determining data about natural products, including: a. performing a 2D NMR technique on an unknown sample; and b. performing a deep learning method on the results of the fast NMR technique.

Implementations of the invention may include one or more of the following. The 2D NMR technique may be a fast NMR technique that screens for suitable fast and NMR pulse sequences at nanomole/picomole sample scales. The 2D NMR technique may employ nonuniform sampling or sparse sampling. The deep learning method may employ a convolutional neural network. The convolutional neural network may be configured to perform dereplication of the sample. The method may further include the step of training the convolutional neural network. The training made dereplicate known compounds both in filtered crude extracts and after purification. The method may further include using an energy-based model and/or a Siamese network to correlate unknown compounds or their moieties with known compounds or their moieties. A Siamese deep convoluted neural network may apply an energy-based model, whereby correlations may be readily performed of unknown compounds or their moieties with known compounds or their moieties, respectively, whereby new leads may be quickly identified without having to perform intermediate and labor-intensive steps of structural and stereochemical determination of known compounds of interest. The deep learning method may perform a step of detection. The step of detection may detect known compounds in filtered VLC fractions, detects known pure compounds, or detects if the value of a suggested compound is compatible with a pattern in a certain spectra. The deep learning method may perform a step of ranking. The step of ranking may determine if a subject sample is more compatible with a first spectrum or with a second spectrum. The deep learning method may perform a step of analyzing. The step of analyzing may determine if an HSQC pattern of a first moiety in a first spectrum appeared in the HSQC of a known category of compounds, while the pattern of a second moiety in the first spectrum was previously solved in a prior analysis. The NMR techniques may include a step of data reconstruction with a combined Poisson Gap and Maximum Entropy Method, giving rise to 2D NMR spectra having an improved signal to noise ratio.

In another aspect, the invention is directed towards a non-transitory computer readable medium, including instructions for causing a computing environment to perform the above method.

Advantages include that, although one may generally still be required to perform the initial steps of assay guided purification to identify potential leads, one need not fully characterize the structures. Rather, one can screen new libraries for other compounds that are related on the basis of the features captured in 2D NMR and can directly identify new leads. Other distinctions and advantages will be understood from the description that follows, including the figures and claims.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE FIGURES

FIGS. 1A-1B illustrate nonuniform sampling, particularly with regard to 2D NMR.

FIG. 2A-2D illustrate various scripts for performing reconstruction.

FIG. 3A shows results of a maximum entropy method alone.

FIG. 3B shows results of a Poisson Gap technique.

FIG. 3C illustrates how a combination of Poisson Gap and a maximum entropy technique is preferred.

FIG. 3D illustrates how both increments and scans have to be increased when the concentration of a sample is lower.

FIG. 4 illustrates a first clustering map.

FIG. 5 illustrates a second clustering map, this clustering map including the labels.

FIG. 6 illustrates a third clustering map.

FIG. 7 illustrates a fourth clustering map, this clustering map including the labels.

FIG. 8A illustrates a first implementation of steps of a method of the invention.

FIG. 8B illustrates a second implementation of steps of a method of the invention.

FIG. 9A illustrates a Fourier transform of sparse sample data (a) and a reconstructed spectra with iterated soft threshold and maximum entropy method (b).

FIG. 9B illustrates a cluster map and various chemical structures of compounds contained therein.

FIG. 10 illustrates a distribution of image number in each category of a training data set.

FIG. 11 illustrates accuracy of the test set based on dimensionality.

FIG. 12 illustrates another cluster map and various chemical structures of compounds contained therein.

FIG. 13 illustrates a cluster map and various chemical structures of compounds contained therein.

FIG. 14 illustrates an increase in accuracy based on an increase in the training and use of the AI, i.e., CNN.

FIG. 15 illustrates results of embedding of unknown HSQC spectra into the clustering map.

FIG. 16 illustrates an enlargement of the local area of the embedding map.

FIG. 17 shows additional details.

FIG. 18 illustrates precision—recall graphs.

FIG. 19 illustrates a sample spectra to which noise has been added.

FIG. 20 illustrates visualizations of features identified and used within the CNN.

FIG. 21 illustrates how the distribution of molecular families on the clustering map evolves.

FIG. 22 shows a 83000 iteration training result.

FIG. 23 illustrates distance of the noised spectra measured against the original spectra of ebracenoid C and hyphenrone I, and how systems and methods according to present principles can still recognize compounds even in the presence of noised spectra.

Like reference numerals refer to like elements throughout.

### DETAILED DESCRIPTION

In more detail, artificial intelligence technologies, such as deep learning, have generated new ways in which to meet the challenges noted above. Compared with conventional machine learning methods, which require a very large number of known training samples for each category, deep learning techniques are designed for small training samples within each category. Moreover, the category numbers for deep learning can be very large and unknown during the training process. Thus, deep learning is an ideal tool by which to analyze and categorize 2D NMR spectra of NPs. For NPs, there are an unlimited number of categories for different compound families, with many being unknown at the present time. Additionally, it is quite common that each category contains less than an estimated 50 different members; in the experience of the current researchers' laboratory working with marine cyanobacterial NPs, this is the case for molecular families such as the curacins, apratoxins and lyngbyabellins.

A desired comparison system for 2D NMR spectra should perform two tasks: the first is ‘detection’ and the second is ‘ranking’. For example, in the ‘detection’ phase, the question to be asked is “are the NMR correlation values of a given compound compatible with spectra A?” This detection is performed by comparing the scalar “energy” of a compound family label with a threshold value. The scalar energy, generated using the Energy Based Models (EBM), is a concept that measures the compatibility of two 2D NMR spectra variables. The system must be trained to generate scalar energy values for 2D NMR spectra in such a way that the value is large when the input spectra are less like that of the compound family. In the ‘ranking’ phase, the question is “considering spectrum B or spectrum C, which is more compatible with a specific compound family?” This is one stage further than detection because the system is trained to produce a complete ranking of all outputs, rather than only present a single best one.

In one implementation, heteronuclear single quantum correlation (HSQC) spectra is recorded using a 2D NMR pulse sequence that measures the heteronuclear coupling between directly bonded nuclei (e.g. ¹H and ¹³C) within an organic molecule. The peaks of those correlated nuclei in the 2D HSQC spectra are generated by detecting coherences connecting states whose total z-angular momentum quantum numbers differ by one order (i.e. single-quantum coherences). In this regard, HSQC spectra are deemed as the fingerprint for each natural product molecule, and thus are highly discriminatory.

This is in contrast to other techniques, such as HMBC or COSY, which do not provide such information, and/or do not provide as clean or neat data, with a minimum of noise. In some cases, such as that of HMBC, too much information may be provided, which prohibits efficient analysis.

Specifically, and in one example, within a 2D HSQC spectra, signals in the direct dimension can be distinguished if they have shifts 0.02 ppm or greater, and in the indirect dimensions if they have shifts of 0.1 ppm or greater. Furthermore, most ¹H chemical shifts occur between 0.5 and 8.0 ppm, whereas in the ¹³C chemical shifts occur between 10 and 175 ppm, which gives rise to the number of distinguishable positions within a 2D HSQC spectrum as 618,750, which is a product of the number of distinguishable shifts in ¹H and ¹³C spectra (375 by 1,650, respectively). Thus, it is clear that the HSQC has great power to discriminate between individual shifts. When one sums this over all protonated carbons in a molecule of 20 carbons with protons attached, the number of potential combinations becomes in the tens of millions, which is considered as “highly discriminatory”.

Another reason that HSQC spectra have been chosen as inputs for training such as Deep Convolutional Neural Network (DCNN) training is that by avoiding detection of double-quantum coherence, the HSQC is usually a clean experiment with relatively few artifacts. By contrast, the heteronuclear multiple bond correlation (HMBC) experiment detects two and three bond correlations by selecting smaller heteronuclear coupling constants (around 5-10 Hz for ¹H-¹³C versus one bond of 125-170 Hz) for double-quantum and zero-quantum transfer. Therefore, while the HMBC experiment has an even larger amount of theoretical information, it is prone to introducing artifacts. In addition, the HSQC when performed with NUS discussed above is a relatively quick and efficient experiment for data accumulation.

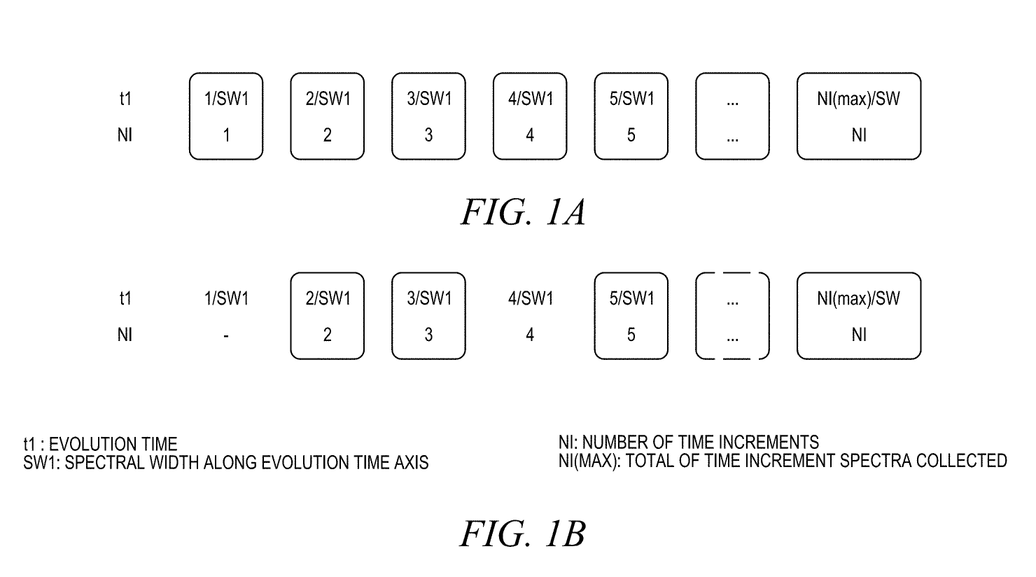

Thus, in one aspect, systems and methods according to present principles, termed “SMART”, cover integration of Fast 2D NMR techniques (Non Uniform Sampling (NUS), etc.) with Neural Network (Deep Convolutional Neural Network (DCNN)) that can quickly dereplicate known compounds and connect or associate unknown compounds with known ones. It is noted in this regard that in conventional 2D spectra, evolution time is varied in successive spectra up to NI(max). See FIG. 1A. Whereas in non-uniform sampling (NUS), data are collected for only a randomly chosen subset of these evolution times, e.g., NI(max)/SW1 (FIG. 1B). In this way the sampling density is reduced to 25% or even 12.5%. However, generally NUS data require special acquisition and processing programs. It is noted that it is not strictly necessary that the 2D NMR be “fast”, but such provides processing advantages.

For example, the use of fast NMR techniques can introduce noise to an HSQC experiment, but at the benefit of a lower experimental time. However, conventional HSQC experiments also encounter noise in HSQC spectra, and in some cases, a fast HSQC experiment gives a cleaner spectrum with a shorter experimental time. When sample quantity is scarce for example, on the order of nanomoles or picomoles, which is quite common for field collected organisms, e.g., toxin containing species, there will be a lower signal-to-noise ratio in HSQC spectra. However, as will be explained in greater detail below, systems and methods according to present principles are particularly adept at ignoring HSQC noise, and still provide outstanding identification and recognition even with small sample sizes. This benefit thus opens the possibility to picomole drug discovery.

In addition, other analysis techniques may be employed, but it is believed that a convolution neural network to be particularly useful in many implementations as will be described below.

In more detail, and as described byLeCun, Bengio, and Hinton, in “Deep Learning”, Nature, 521, p. 444 (28 May 2015), “Deep learning methods are representation learning methods with multiple levels of representation, obtained by composing simple but nonlinear modules that each transform the representation at one level (starting with the raw input) into a representation at a higher, slightly more abstract level. With the composition of enough such transformations, very complex functions can be learned. For classification tasks, higher layers of representation amplify aspects of the input that are important for discrimination and suppress irrelevant variations.” And from Schmidhuber, “Deep learning in neural networks: an Overview”, Neural Networks 61, p. 85-117 (2015): “A standard neural network (NN) consists of many simple, connected processors called neurons, each producing a sequence of real valued activations. Input neurons get activated through sensors perceiving the environment, other neurons get activated through weighted connections from previously active neurons. Some neurons may influence the environment by triggering actions. Learning or credit assignment is about finding weights that make the NN exhibit desired behavior, such as driving a car. Depending on the problem and how the neurons are connected, such behavior may require long causal chains of computational stages, where each stage transforms (often in a nonlinear way) the aggregate activation of the network. Deep learning is about accurately assigning credit across many such stages.” Systems and methods according to present principles use deep learning as defined above, and in particular with respect to neural networks, and even more particularly with regard to convolutional neural networks.

In one implementation, the SMART includes (1) a Fast 2D NMR program for a HSQC (Heteronuclear Single Quantum Correlation) adapted nuclear magnetic resonance (NMR) instrument, with the data reconstructing methods optimized to quickly yield 2D NMR spectra; (2) a Siamese DCNN applying the Energy-Base Model (EBM) trained with a database containing over 4000 2D NMR spectra to generate embedding maps. The 2D NMR data of newly isolated unknown compounds are collected by applying the Fast 2D NMR program that saves up to 75% or more time than conventional 2D NMR programs.

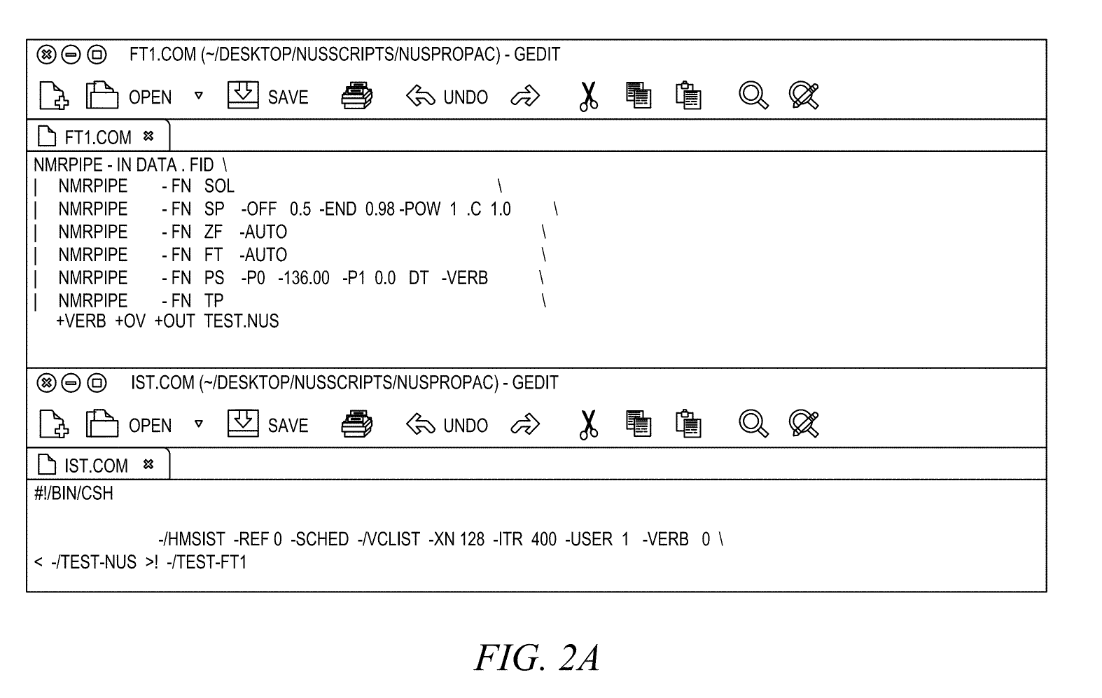

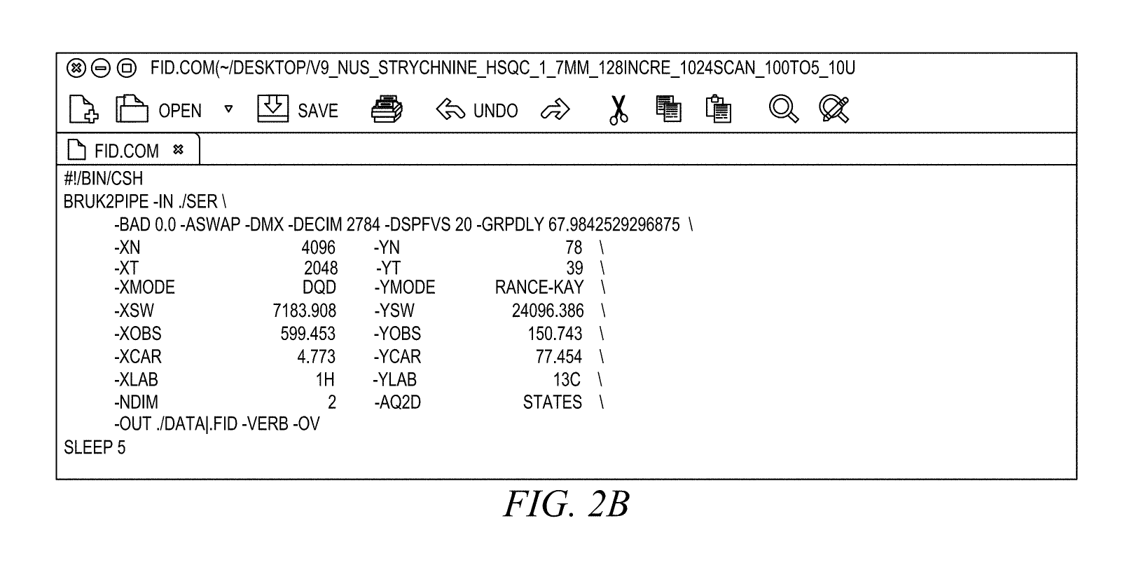

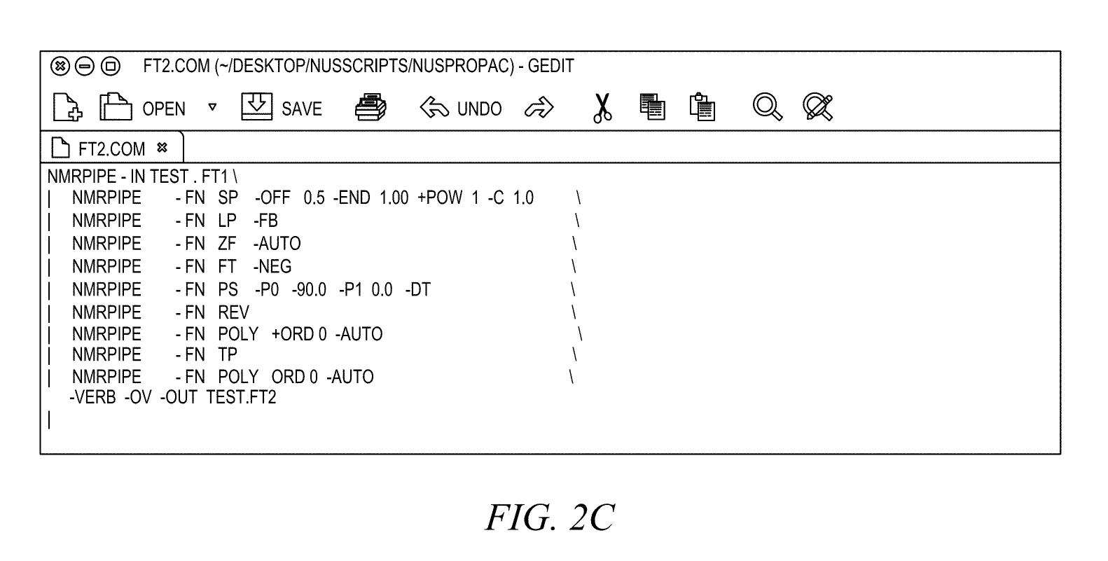

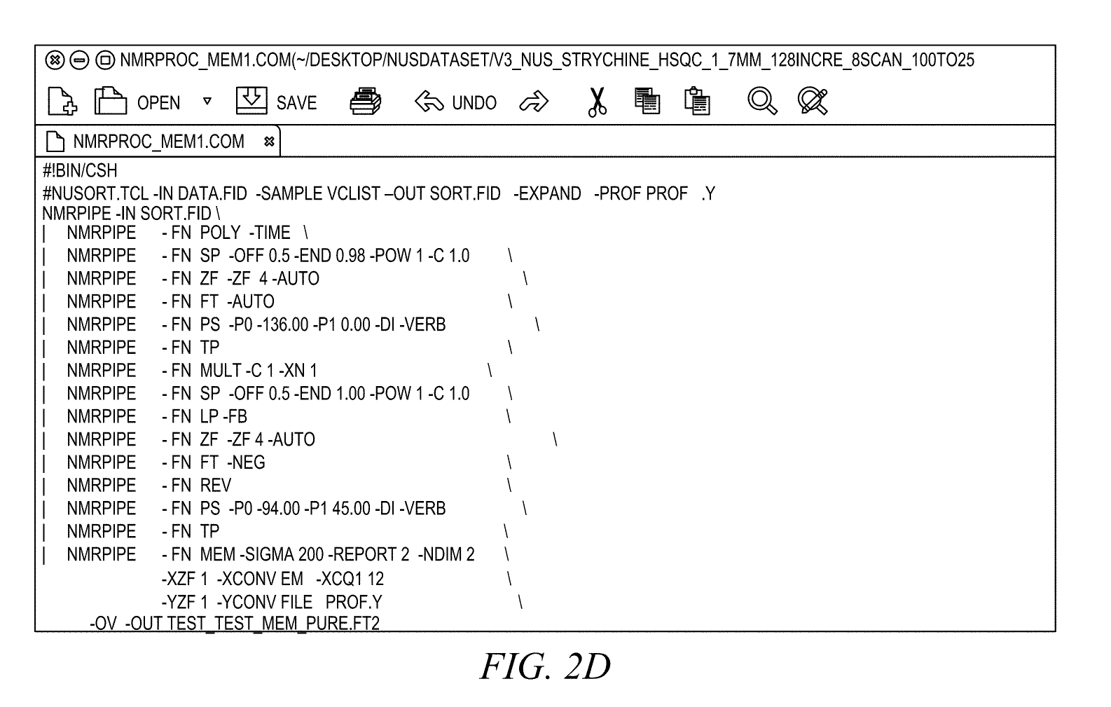

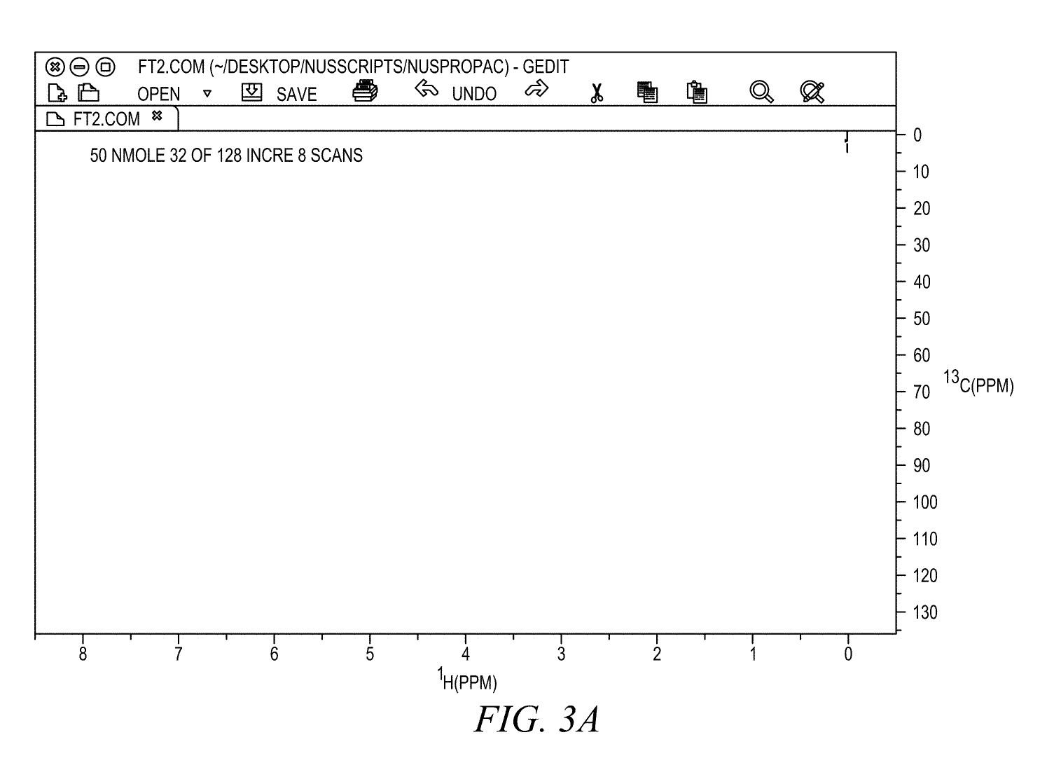

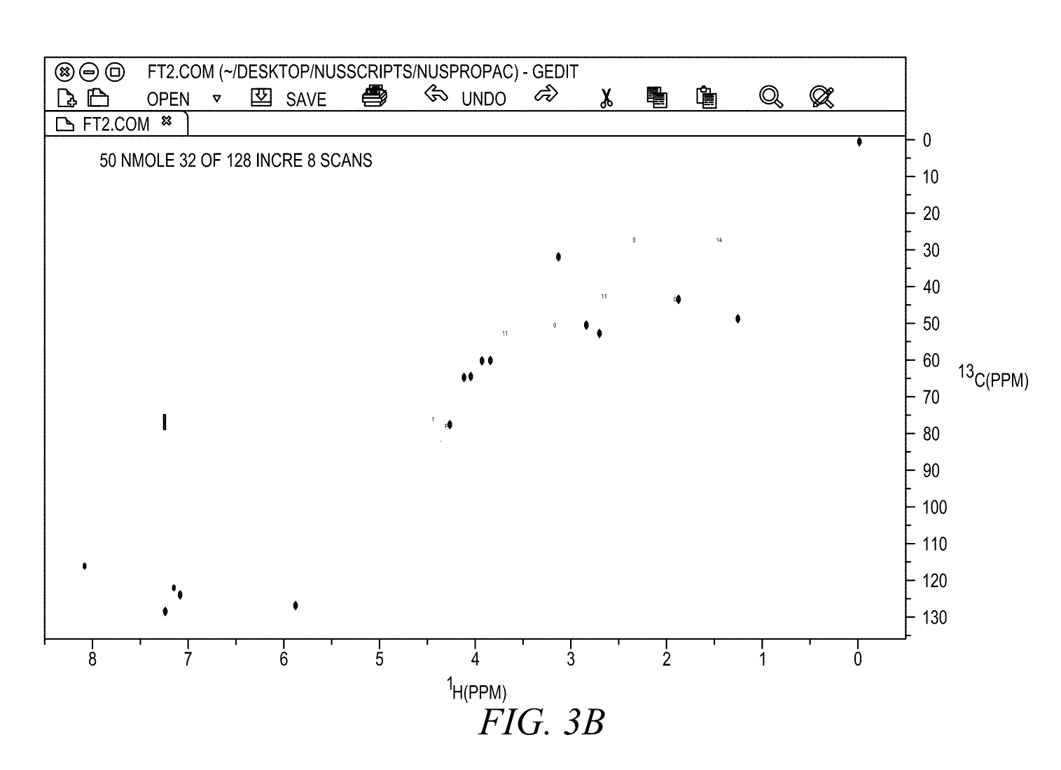

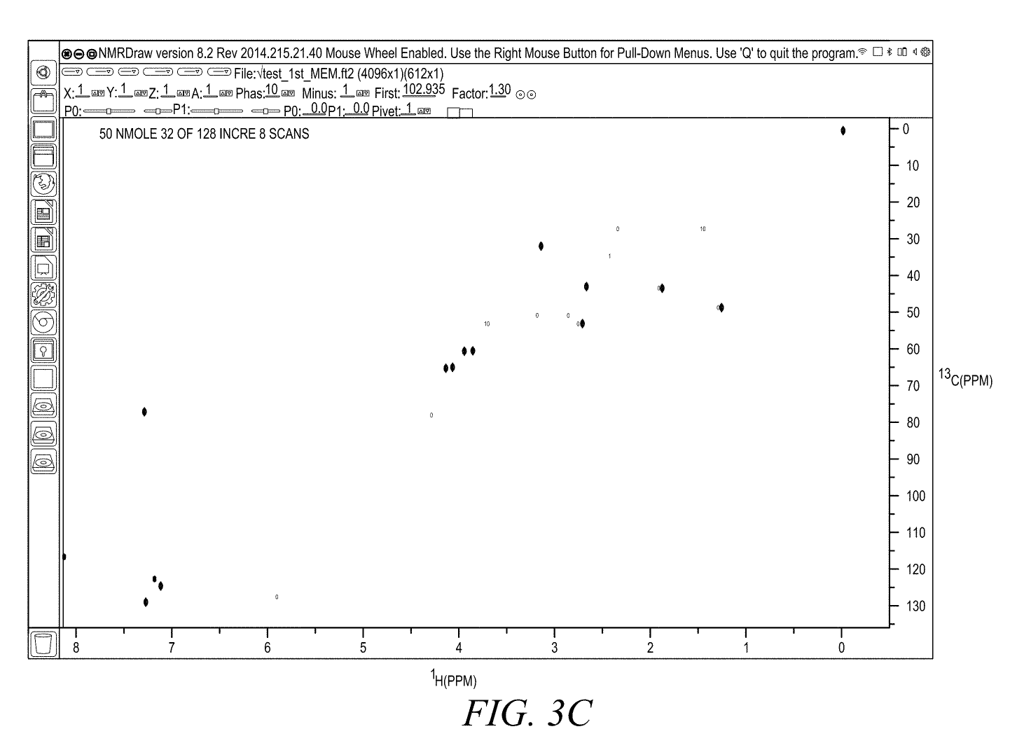

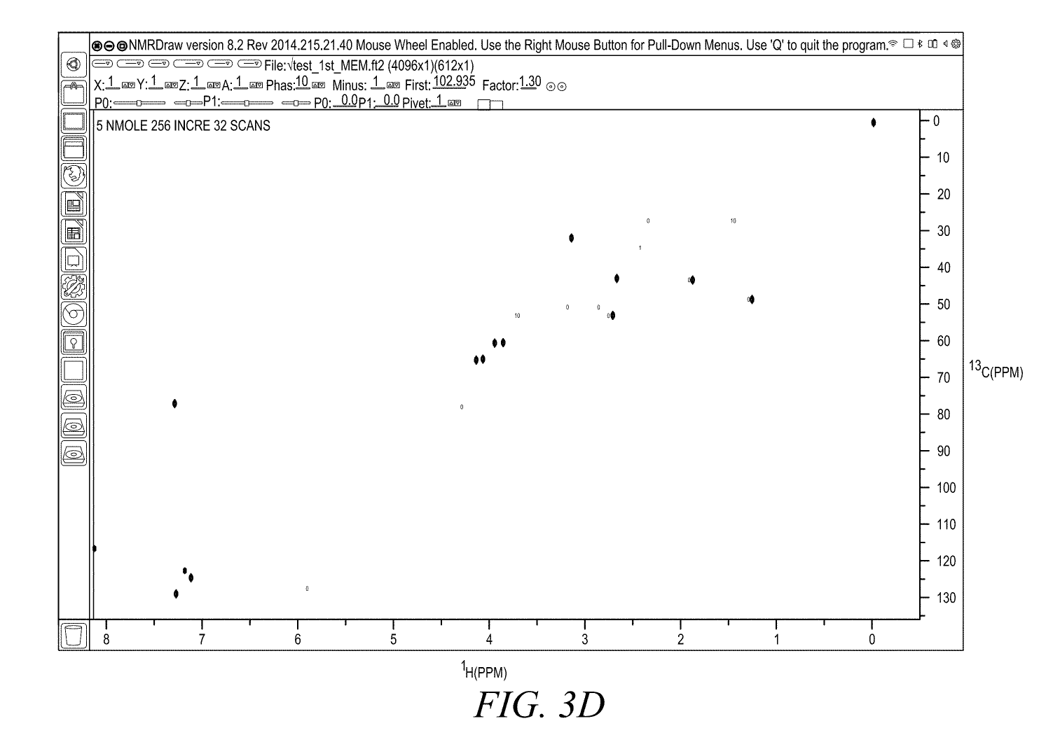

The collected data is then subject to data reconstruction with a combined Poisson Gap and Maximum Entropy Method, giving rise to 2D NMR spectra. See FIGS. 2A-2D for exemplary scripts. As can be seen in FIGS. 3A-3B-3C, a combined Poisson Gap and maximum entropy method is superior to just a maximum entropy or Poisson Gap method individually. As may be seen in FIG. 3D, if the concentration of product is lessened, both the number of increments and the number of scans may have to be increased.

Those generated 2D NMR spectra, or just conventional 2D NMR spectra, can then be provided to the DCNN for spectral detecting and ranking purposes. The output of those results are embedded in a map, with nodes representing each spectra or compound. By comparing these embedding maps with the embedding maps of the 2D NMR spectra database, the unknown compounds are either dereplicated as known compounds or accurately categorized into existing compound families. This allows an appreciation of the great structural diversity of chemical compounds, and indirectly, streamlines the discovery pipeline for bioactive ones. In this regard, the method allows a mapping of chemical diversity as visualized through their HSQC spectra.

The subsequent description describes certain aspects of the DCNN process.

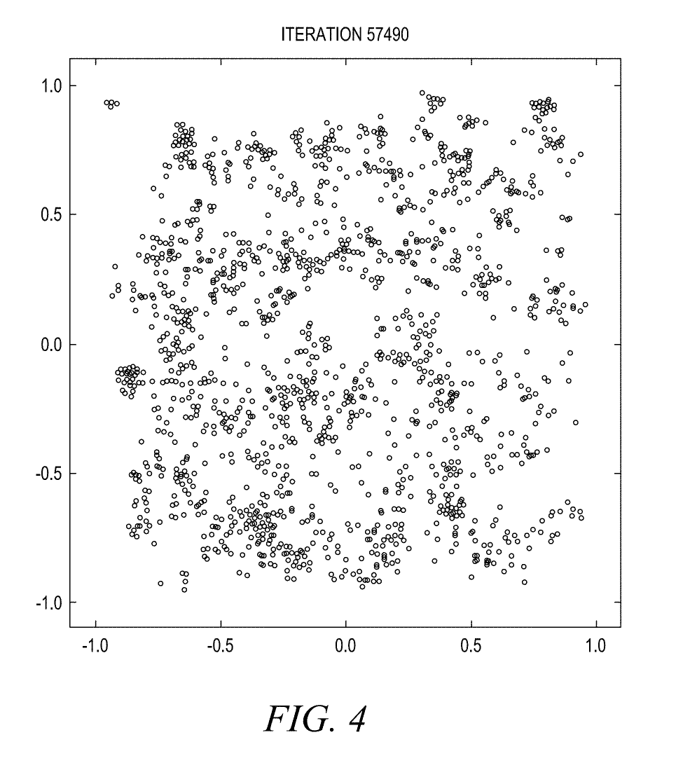

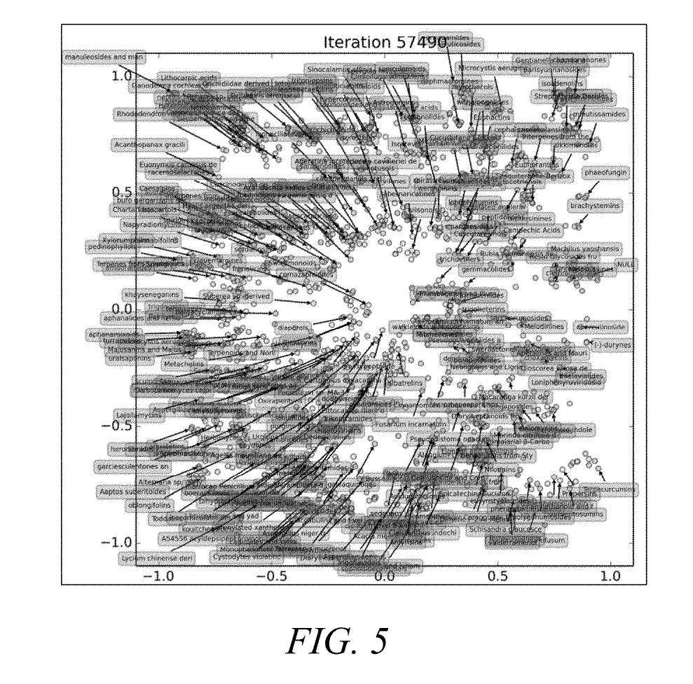

In FIGS. 4 and 5, which are statistical maps for the training results of 4110 HSQC spectra, and which are equivalent, compounds that share similar structures are clustered together. Compounds that were not subject to the training steps were tested for recognition by the SMART system. Spectra were obtained from the Supporting Information pages of the Journal of Natural Products as described below. The test process does not require rerunning the training steps. In other words, within a few seconds, the DCNN is smart enough to categorize compounds into compound families that it learned during the training steps.

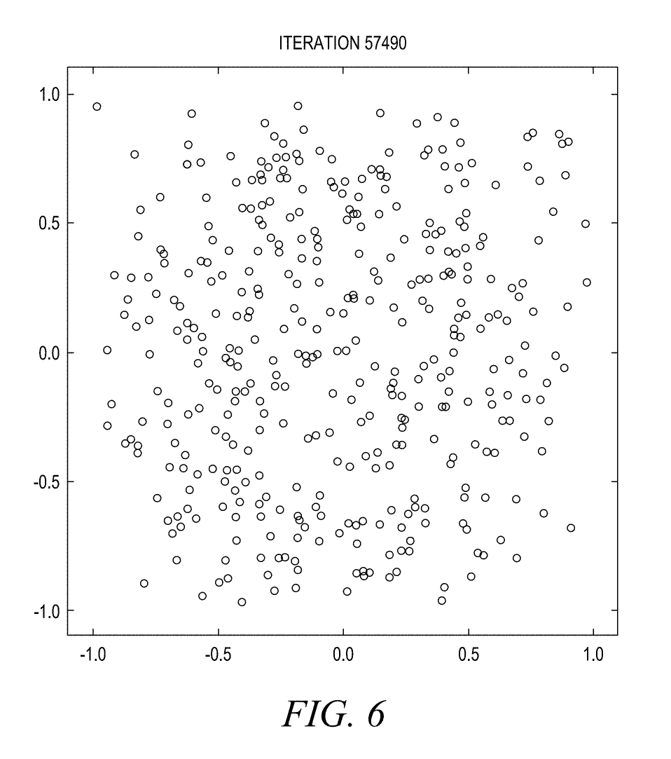

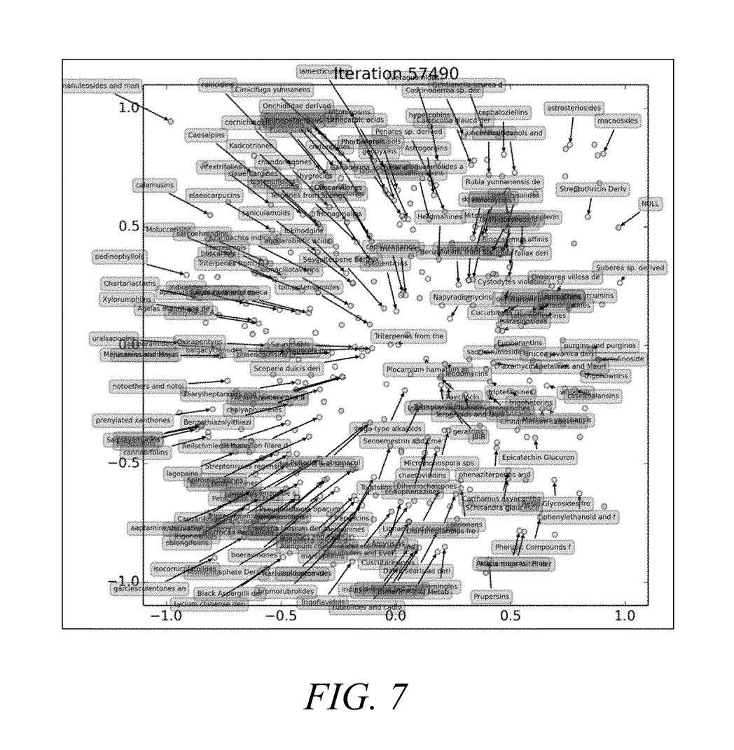

In FIG. 6 and FIG. 7, which are also equivalent, HSQC spectra that were not subject to the training steps were tested by the SMART system and method of recognition, and then these were used to give rise to a statistical map. These figures represent statistical maps for the test results of around 100 HSQC spectra. To give an example, as indicated by the red circle of FIG. 6, and in a corresponding region of FIG. 7, “manuleosides” are in the same regions.

As noted above, previously-reported 2D NMR comparison methods use 2D NMR techniques like HSQC that sample all increments in the indirect dimension. Although systems and methods according to present principles can work on conventional NMR spectra as well, it can work equally well with HSQC spectra wherein non-uniformly sampled increments are obtained in the indirect dimension during 2D NMR acquisition, a process which saves time and provides high quality spectra. Currently, computer-aided structure elucidation (CASE) (by, e.g., ACD/Labs, etc.) is largely based on applying a least-squares regression approach which compares NMR chemical shifts and are generally insufficient to tackle solvent effects, artifacts, or other problems noted above. In addition, other methods of analysis of 2D NMR generally require multiple methods, are significant iterative and use heuristic analysis to provide results.

However, one exemplary analysis technique, DCNN, is image recognition based and is not associated with the difficulties above in that it does not require chemical shift comparisons.

In more detail, a Small Molecule Accurate Recognition Technology (“SMART”) prototype was created by integrating the benefits of NUS NMR together with the advances in AI to reshape Natural Products Research (NPR) and thus improve the efficiency of NP drug discovery research. To initiate SMART, a database of training sets containing over 4110 experimental 2D HSQC spectra, deriving from this same number of compounds, was provided to a DCNN employing a Siamese Architecture as will be described below. Also presented in the manifestation of the SMART is a Training Embedding Map (TEM). To further demonstrate the ability of SMART to recognize and help identify new NPs, NUS HSQC spectra were collected for several nonribosomal peptide synthetase (NRPS) derived NPs that had been isolated from a marine cyanobacterium. These were entered into SMART with subsequent observation of their placement within the test embedding map, and these were accurately identified to reside within the ‘viequeamide’ subfamily of NPs.

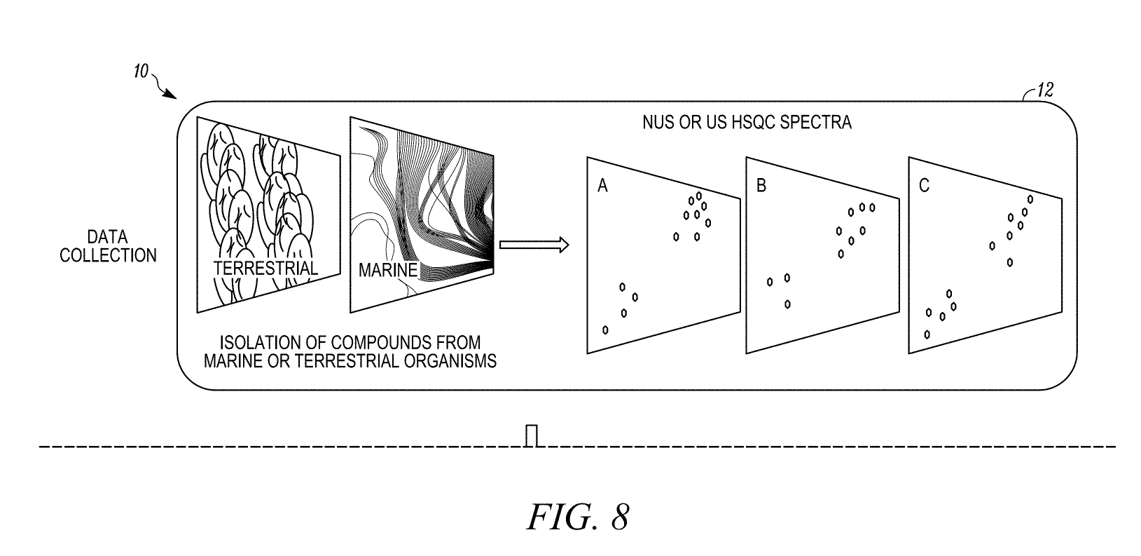

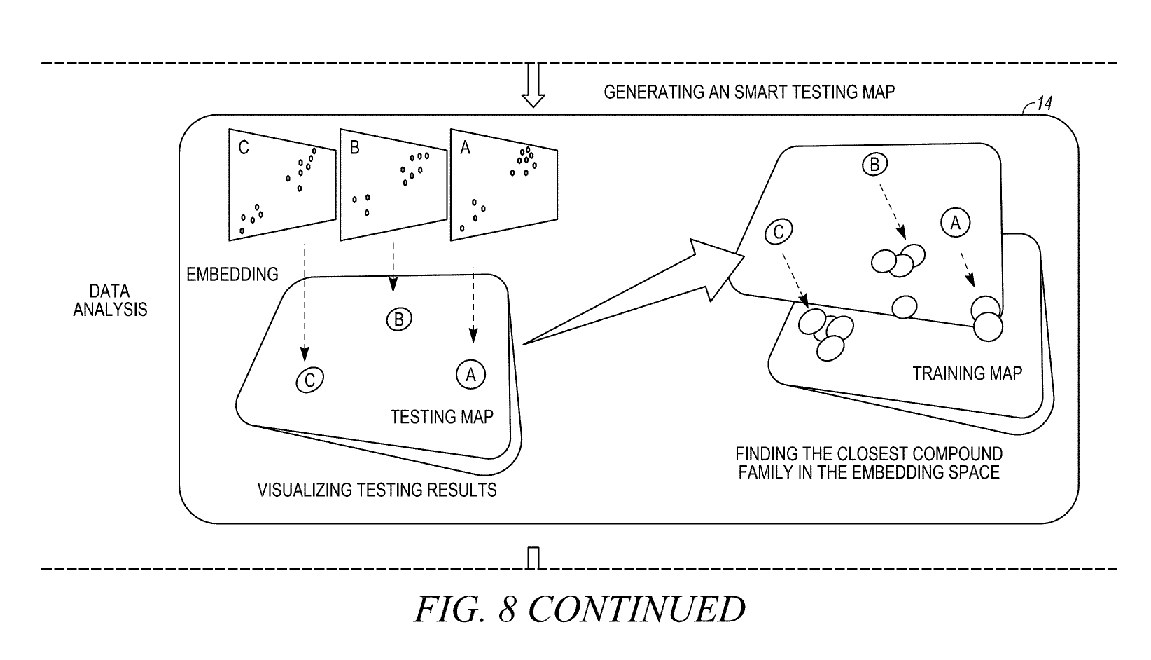

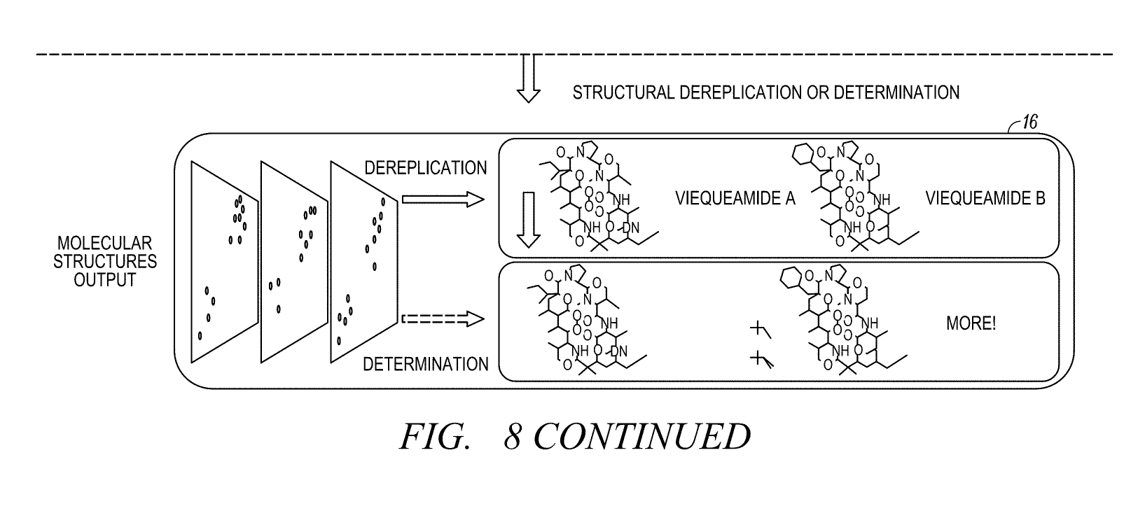

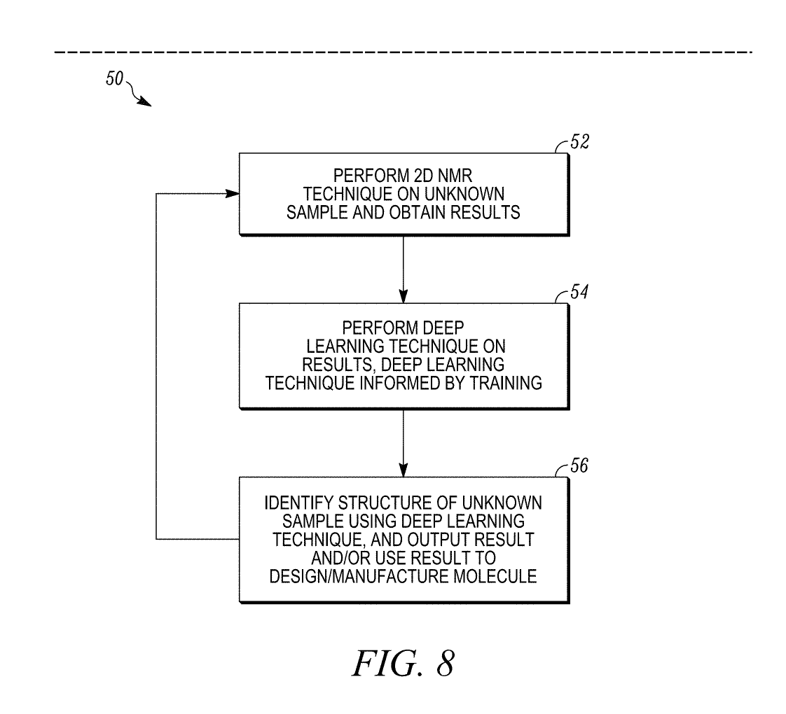

SMART is a user-friendly, unbiased, AI-based dereplication and analysis tool that utilizes 2D NMR data to rapidly associate newly isolated NPs with their known analogues. SMART has been designed to mimic the normal path of experiential learning, in that additional 2D NMR spectral inputs can be used to enrich its database and improve its performance. In short, SMART will become an experienced associate to natural products researchers as well as other classes of organic chemists. The SMART workflow, in one implementation, necessitates three steps, 1) 2D NMR data acquisition by NUS HSQC pulse sequence, 2) 2D NMR spectral analysis by DCNN, resulting in a projection of the spectra into a similarity space of NPs, and 3) molecular structure output by the user. This process gives users rapidly access a well-organized map of structurally determined NPs, and helps ensure that SMART's insights are chemically rational. In this regard, the SMART capitalizes on the enormous wealth of molecular fingerprints, namely 2D HSQC spectra, built over the past four decades, and, reciprocally, the 2D HSQC spectral database will experience a non-linear expansion of this dataset as a result of SMART's application. FIG. 8A illustrates the workflow of SMART using, as an example, the viequeamide NPs.

In more detail, the workflow which develops from a practical application of SMART begins with recording the NUS HSQC spectrum (step 12) for a pure small organic molecule; in the case of NPR, this is a substance extracted and purified from an organism of interest. As noted conventional HSQC spectra may also be employed. A testing map is then generated (step 14), which involves data analysis and finding the closest compound family in the embedding space. The HSQC spectra are embedded into an AI clustering map which results from the training of the SMART and particularly the DCNN. The goal is to find the closest structurally resembling compound family to the unknown compound in the AI clustering map. Finally, a step is performed of structural dereplication or determination (step 16), in which a particular compound may be de-replicated or determined as being statistically similar to another compound. An output is then provided to the user, which can be displayed or used as part of a drug discovery experiment, where the output is the molecular structure.

FIG. 8B is a flowchart of another implementation of a method according to present principles. In a first step, a 2D NMR technique is performed on an unknown sample, and a result is obtained (step 52). In a second step, a deep learning technique is performed on the results from the 2D NMR, the deep learning technique typically informed by a training, e.g., a neural network or a convolutional neural network. In a third step, the structure of the unknown sample is identified using the deep learning technique, and a result is outputted (step 56). The deep learning technique is typically trained, and then when it is exposed to a new 2D NMR spectrum, the deep learning technique can determine the relationship of its HSQC to those of a library of structures. It is noted that this is not simply a matter of matching templates. In many cases small variations can lead to misinterpreted results if only templates were used. By using deep learning techniques, and in particular a convolutional neural network, compounds may be identified, e.g., if the convolutional neural network has been trained on them. Moreover, similar compounds can also be identified, and if one point of the spectrum is slightly out of place compared to others, it will be placed in nearby space to show it is related. Simple template matching is not able to determine these types of similarity; however, the present systems and methods, employing deep learning in convolutional neural networks, are able to do so.

It is noted here that a small molecule is here defined as one whose transverse relaxation time constant (T₂) is on the same order of magnitude as its longitudinal relaxation time constant (T₁) when dissolved in liquid solution. In other words, the nuclear spins of a small molecule should keep synchronized between 10⁸ to 10⁷ Larmor precession cycles during a liquid state 2D HSQC experiment. Nevertheless, the SMART concept is not inherently confined to small molecule NUS NMR spectra, considering the ability of NMR to structurally characterize molecules of many sizes and types. NUS HSQC experiments are highly advantageous for small molecule structure elucidation compared with conventional pulse sequences due to their rapid acquisition, few spectral artifacts, and intrinsically high resolution. Nevertheless, as discussed below, conventional 2D HSQC spectra can be provided to the AI system and spectrum recognition achieved. In fact, the initial database of HSQC spectra that were compiled to train the SMART system were acquired in this manner.

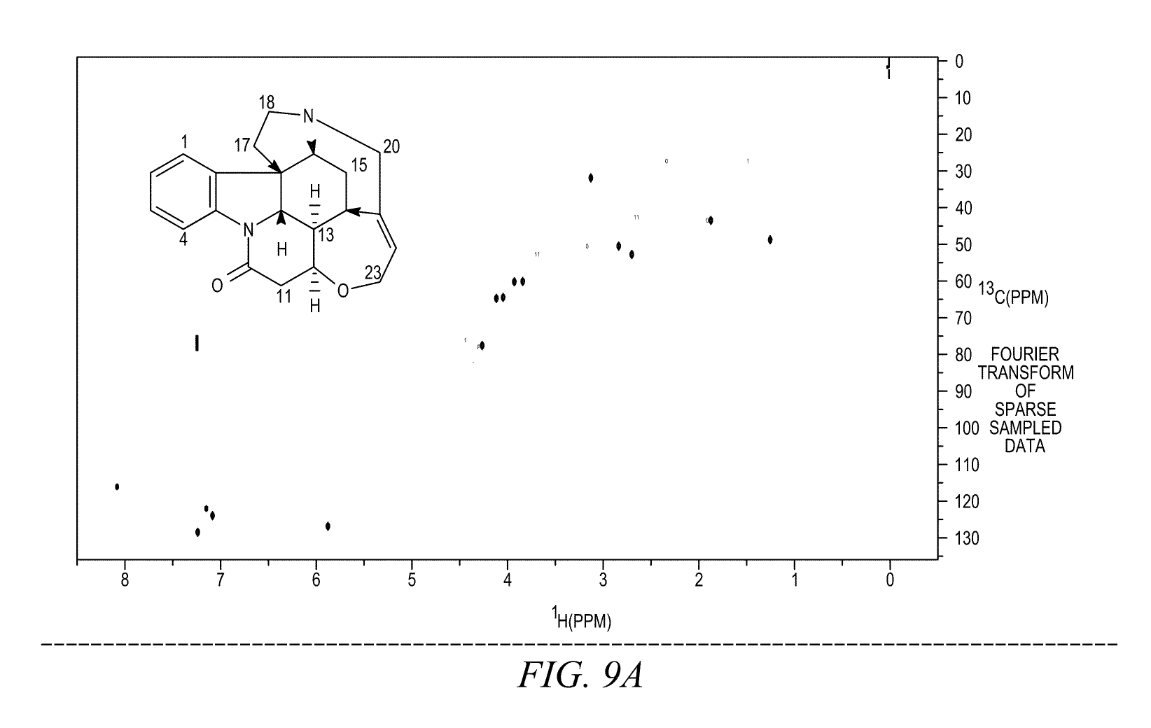

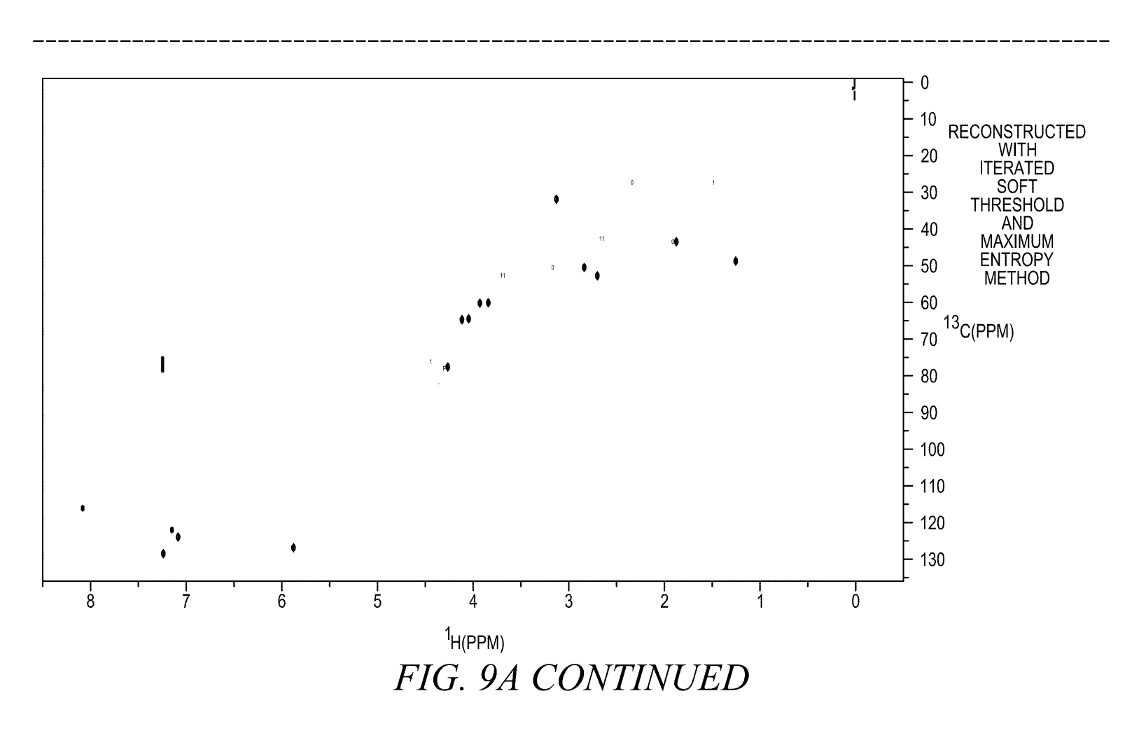

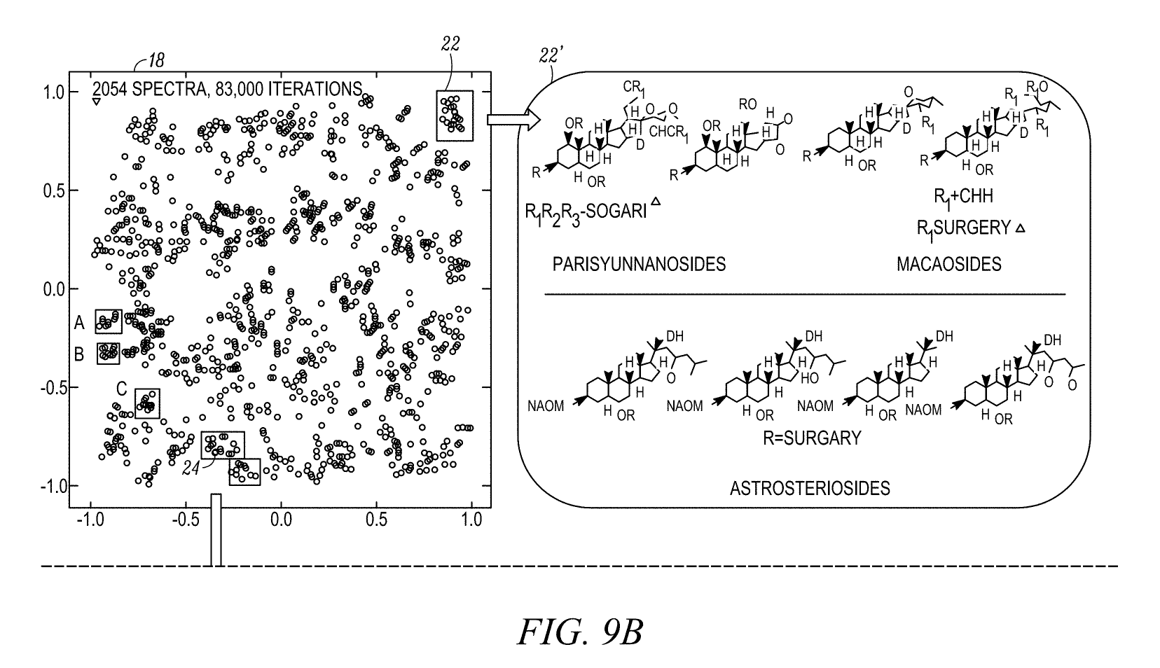

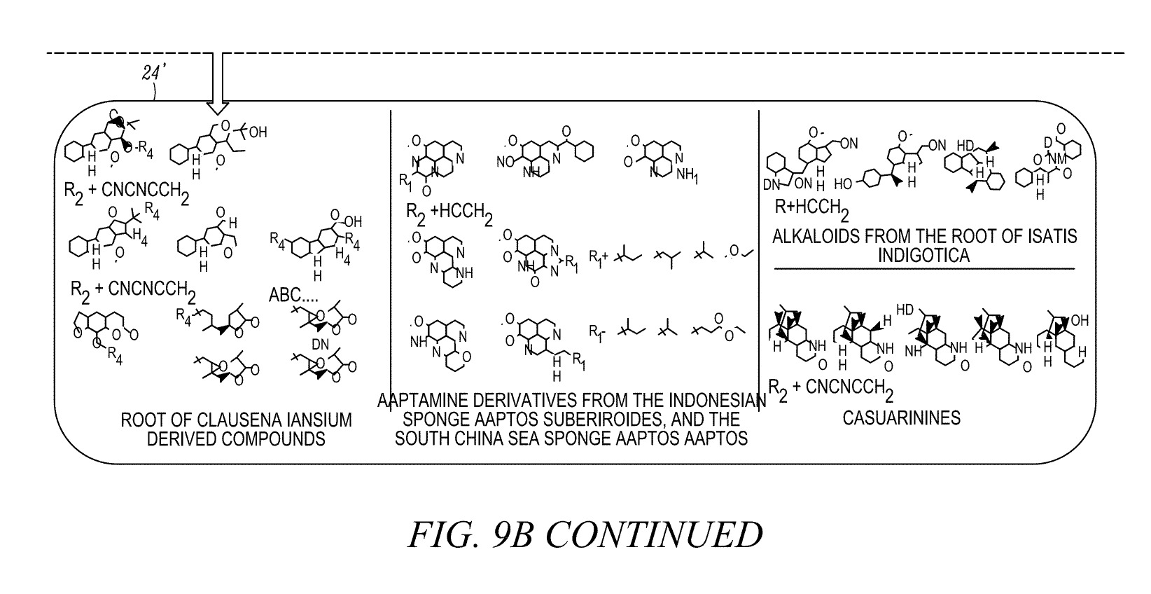

Due to lower sampling density, NUS HSQC requires alternative approaches to convert the time domain of the collected signal into visual spectra of the frequency domain, and thus methods other than the Discrete Fourier Transform are required. Towards this end, Iterated Soft Thresholding (IST), followed by the Maximum Entropy Method (MEM), were applied on the NUS data collected using strychnine as an NMR model compound. See FIG. 9A. In order to find convergence in local minima, the Lagrange multiplier was applied to weight the regularization function, the L₁ norm, in the IST routine. Previous studies have shown that IST is superior to Maximum Entropy Reconstruction (MaxEnt) (not to be confused with MEM) in NUS NMR data reconstruction, owing to the simplicity of IST with fewer adjustable parameters and the resultant ease of application. Admittedly, IST suffers slower convergence over MaxEnt for spectra with a high dynamic range. However, it has been shown that changing the step sizes in IST can achieve visualization of the final spectra indistinguishable to those reconstructed by a well-tuned MaxEnt process. The MEM can then be applied after Fourier Transformation of the IST reconstructed data in the direct dimension, an improvement resulting from the fact that MEM is biased towards the enhancement of smaller line widths. With the model compound, the signals of the methylene hydrogens (3.11 ppm and 2.67 ppm) adjacent to the carbonyl group of strychnine were visibly strengthened after sequentially applying IST (400 iterations) and MEM (3 iterations) compared with application of IST (400 iterations) with Linear Predictions (LP) during data reconstruction of the non-uniformly sampled 2D NMR spectra.

As noted, in one implementation, systems and methods according to present principles can use a deep learning method that is based on a Siamese neural network architecture. A Siamese network is a pair of identical networks that are trained with pairs of inputs that are mapped to a representational space where similar items are near one another and different items are far; that is, it produces a clustering of the input space based on a similarity signal. In this case, it maps the input HSQC spectra into a lower dimensional space where HSQC spectra are clustered. For SMART5: positive pairs equaled 5982, negative pairs equaled 2103476. For SMART10: positive pairs equaled 3787, negative pairs equaled 410718. The number of pairs grows with an order of O(n\hat{ }2). During the training, a minibatch of pairs (size 200) was generated, so that 100 pairs were randomly chosen from the positive pair set, and 100 from the negative pair set. The 100 pairs are resampled every time.

This explains one reason why a Siamese network is particularly appropriate for this task. To train a deep network, typically on the order of a million examples are used. The advantage of the Siamese network is that they amplify a small data set by training on pairs. A second reason why Siamese networks are appropriate is that a cluster space was required, rather than a classification of compounds into families. If the system were simply trained to take an example and classify it, this would not be appropriate for new compounds for which the category is unknown. The cluster map generated by SMART places the new compounds into a similarity space.

The HSQC spectra are inherently a visual input, and so the network used in the Siamese network was initialized using a DCNN that had been pre-trained in a large-scale visual task, classification of images from the ImageNet dataset. Convolutional neural networks (CNNs) are currently the best method for image processing in the computer vision community, and have revolutionized the field of computer vision. Like standard neural networks, they are trained by backpropagation of error. CNNs are structured to learn local visual features that are replicated across the input, hence the name “convolutional.” The local maximum of these features are then input to another layer that learns local features over the previous layer of features, and this process repeats for several layers. By using the local maxima of feature responses over nearby locations in the input, the network will generalize to patterns that are shifted in the (x, y) plane of the spectra, i.e., it becomes translation invariant. Thus the network is hierarchical, like the mammalian visual system, and learns more and more abstract features in deeper layers of the network. In a Siamese network, the final layer is not trained to classify the inputs; instead, a set of units are trained to give similar patterns of activation for similar inputs (as given in the teaching signal) and different patterns of activation for inputs that are labelled as different. Hence they produce a clustering in the space of unit activations.

Hence molecules that are similar will be mapped to nearby locations in the output space. If the network generalizes well, it will place novel molecules near known ones that have similar NMR spectra. This allows the system to rapidly identify candidate known molecules that may have similar biological action to the novel molecule, allowing the user to search through a small subset of known molecules for similar compounds. In an initial approach, two output units were used, and they thus defined a two dimensional clustering of the molecules that is easily visualized. As a particular example, the SMART was able to identify some intermediate saponins between polysaccharides and terpenoids.

### Network Training and Results Mapping

The neural network may be trained using Stochastic Gradient descent by computing gradients

$$\frac{\partial }{\partial W} \hat{L}$$

for each minibatch. In one implementation, an adagrad update rule was used. To speed up the training, batch normalization may be employed which reduces internal covariance shift by standardizing features on each layer. Using batch normalization, in one implementation, the network trains 5 times faster. In more detail, initial experiments on SMART5 with k=2 dimensional embedding were run for 100k iterations on an Amazon EC2 g2.2×large instance (using NVIDIA GRID K520 GPU) which required 8 days. Using batch normalization, the time was reduced to 28 hours. Final experiments on SMART5 were run on an Nvidia Titan X (Maxwell), and the number of iterations was limited to 15k; this was completed in 3-4 hours.

Upon training the multilayer DCNN, the datasets are divided into three subsets; the training set containing 80% of spectra that are used to adjust the weighting, the validation set containing 10% of data used for monitoring, and the test set containing the remaining 10% that were used as a proof of concept. The validation and test set includes HSQC spectra that are not applied during the training process. The error on the validation set is monitored so as to ensure that error on the unseen portion of data reduces once the training process is initiated. The test results are then embedded in the clustering map, sometimes termed a training embedding map or TAM, to locate their nearest neighbors within the TAM. In this way, the test HSQC spectra are correlated and matched with other compounds of structural similarity shown in the TAM.

For quantitative analysis of the approach the top N error was measured: for each sample from the test set the closest N labels in embedded space were predicted, and if one was correct, the sample marked as correctly classified. Results for the best run are reported in the table below, which shows the accuracy calculation result applying cross validation. In this, as noted above, the data set was divided into three sets, namely, a training set, a validation set, and a test set, containing various percentages, as noted, of the data set. The three sets later underwent cross validation.

| | |
|---|---|
| Validation Error | Test Error |

| | mean | std | mean | std |
|---|---|---|---|---|
| Top 1 | 17.718 | 1.9807 | 13.6791 | 2.3517 |
| Top 5 | 31.844 | 3.1022 | 28.0400 | 3.7400 |
| Top 10 | 39.8543 | 3.0116 | 35.0047 | 3.3640 |
| Top 20 | 49.4660 | 3.3240 | 45.3239 | 3.9603 |

So that the results are visually comparable, the outputs of both the training and the test results of SMART are visualized in a two dimensional map. Each node represents an HSQC spectrum processed by SMART. The node colors designate compounds originating from different research articles. When available, the node labels are the compound names; otherwise, the labels are for the organism from which the compound derives. A smaller dataset containing 900 HSQC spectra was first mapped into node clusters with 4800 training iterations, and subsequently, the larger dataset was fed to SMART. With an augmented training dataset containing 900 spectra, a total of 83,000 iterations were performed at which point the node clusters manifested. The tight structural similarity between the TAM and the test embedding map, sometimes termed an embedded clustering map or TEM, was shown by the close correspondence in the location of nodes between them.

Structurally similar NPs were found to form distinct clusters, both in the TAM and TEM. Three clusters are discussed here to highlight this distinct clustering of different molecular entities, one for a terpenoid family and two for an aromatic alkaloid group. See the clustering map 18 of FIG. 9B.

A cluster 22 comprised of 40 nodes (see the upper right box) was found to contain three saponin variants together with other corresponding triterpenoids. See the inset indicating particular similar compound diagrams of box 22′. The three saponin variants, parisyunnanosides, macaosides, and astrosteriosides, are of different geographic origins and are produced by organisms from different biological orders. The parisyunnanosides were isolated from the rhizomes of the terrestrial plant Paris polyphylla Smith var. yunnanensis originating in Lijiang, Yunnan Province, China. The macaosides were obtained from the aerial parts of the terrestrial plant Solanum macaonense collected in Kaohsiung, Taiwan. Finally, the astrosteriosides were isolated from the marine starfish, Astropecten monacanthus found in Cat Ba, Haiphong, Vietnam. The parisyunnanosides have been reported to be toxic to leukaemia cells whereas the macaosides and astrosteriosides have been found to be anti-inflammatory.

Referring to the same figure, a second cluster 24 consisting of 42 nodes was comprised of poly-heterocyclic aromatic alkaloids. Within this cluster, and referring to the box 24′, there are four major molecular families with the heterocyclic components being a pyrrole, imidazole, pyridine, or pyrazine, or a combination of these. Notably, several congeners of aaptamine, isolated from two varieties of Aaptos species collected in different geographic locations, are found in this cluster. A third cluster was composed of phenolic amides known as the teuvissides (box with arrow pointing to it in upper left box), which are anti-hyperglycaemic compounds isolated from Teucrium viscidum collected in Fujian Province, China.

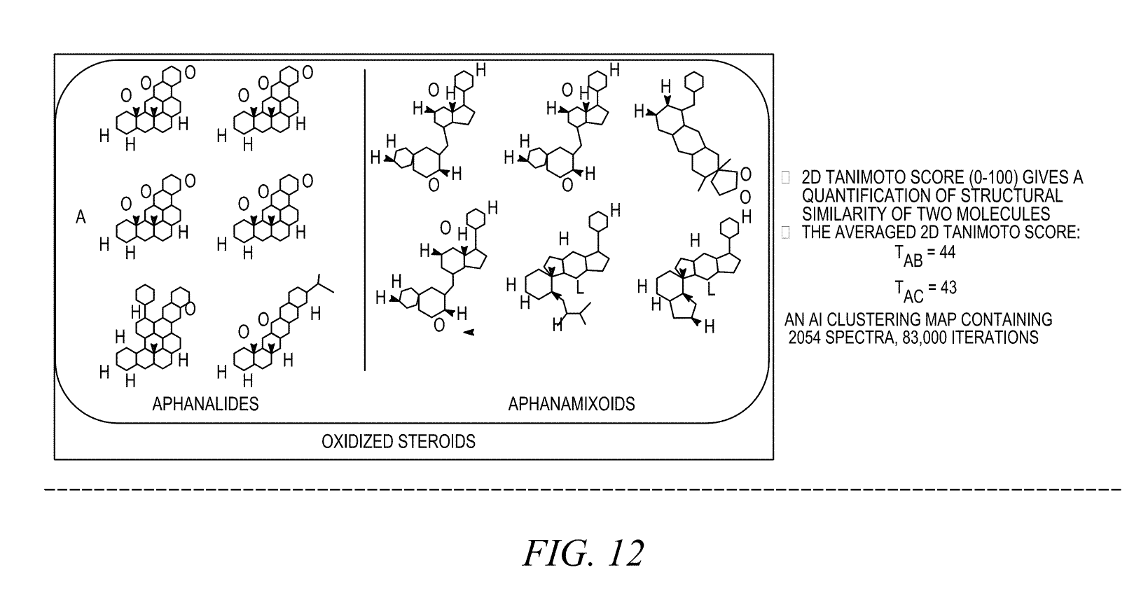

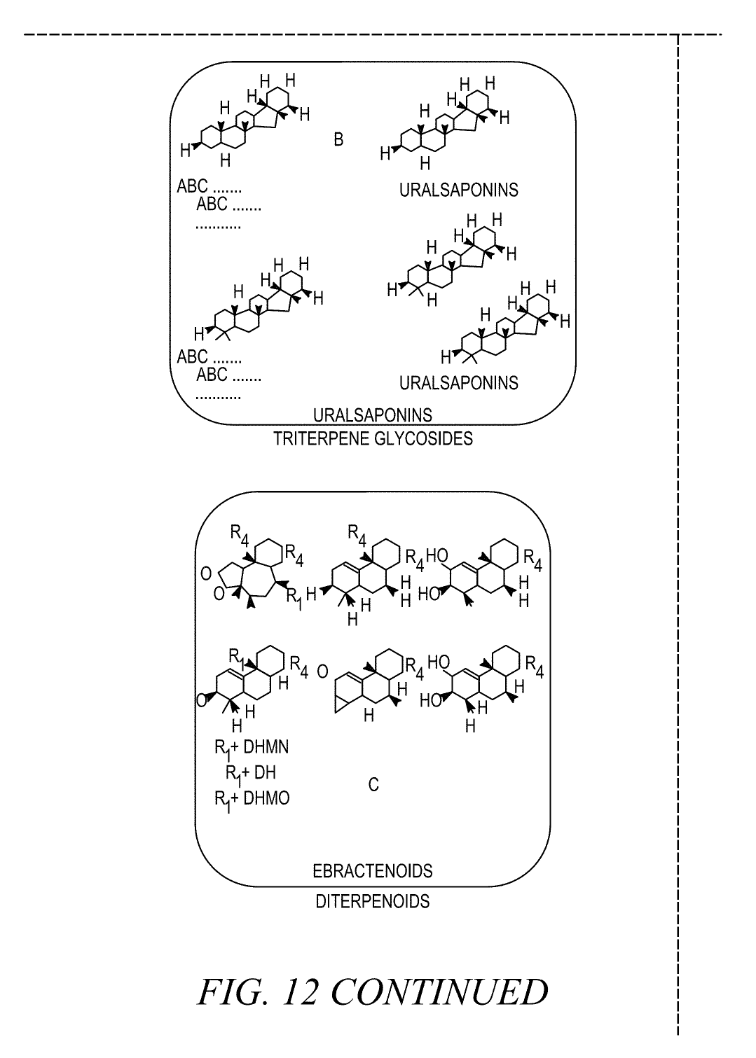

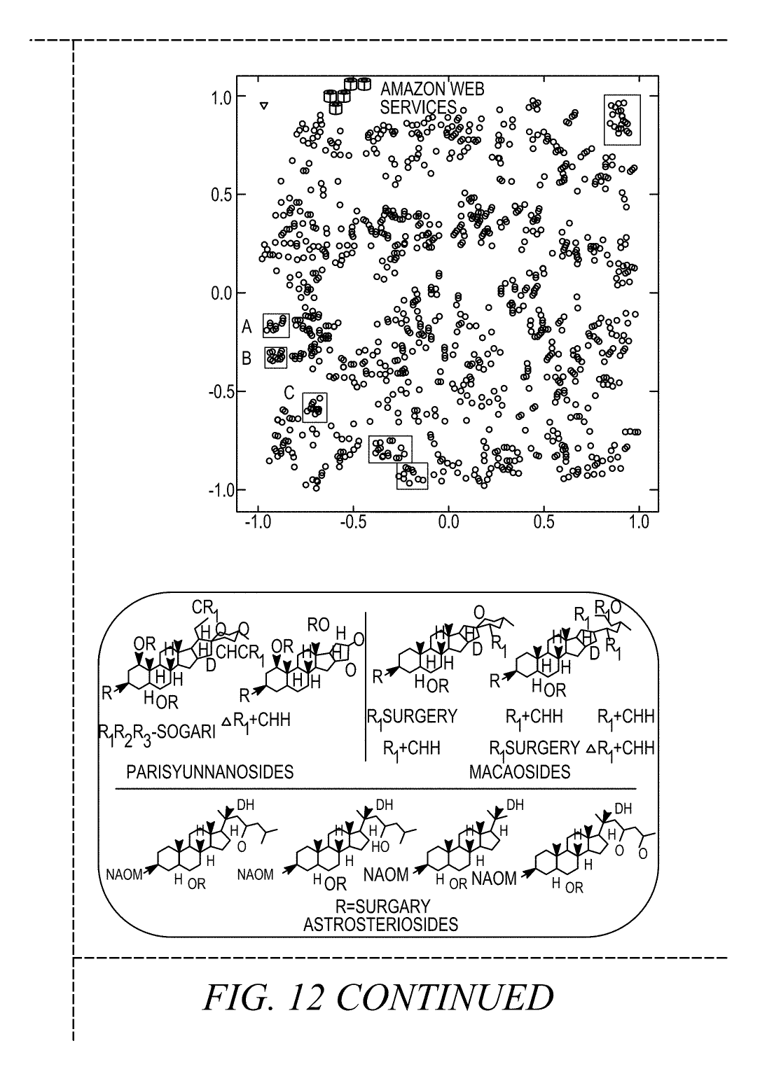

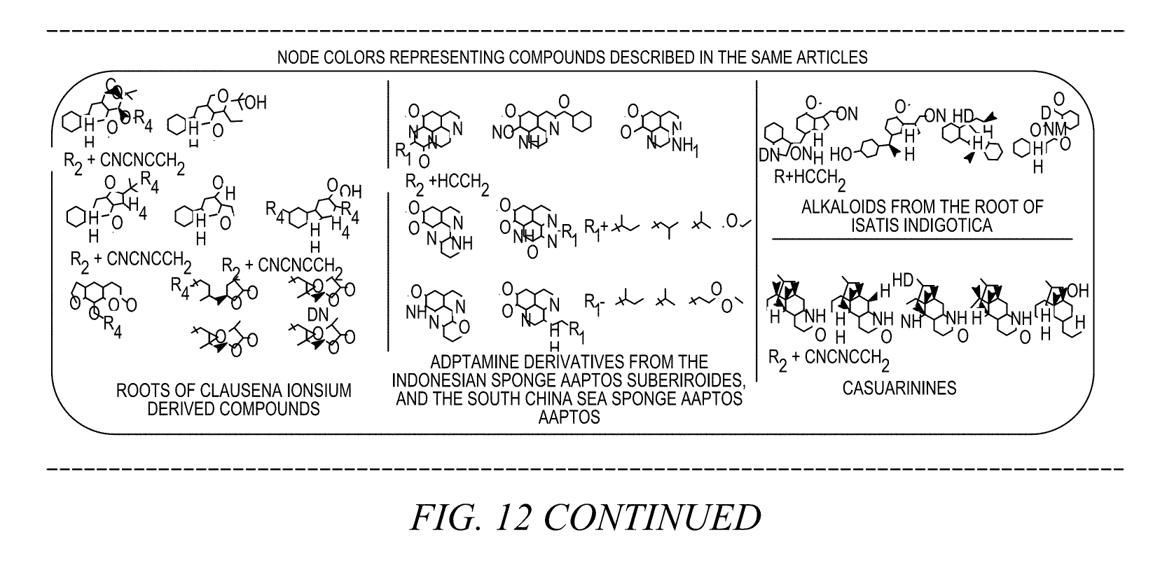

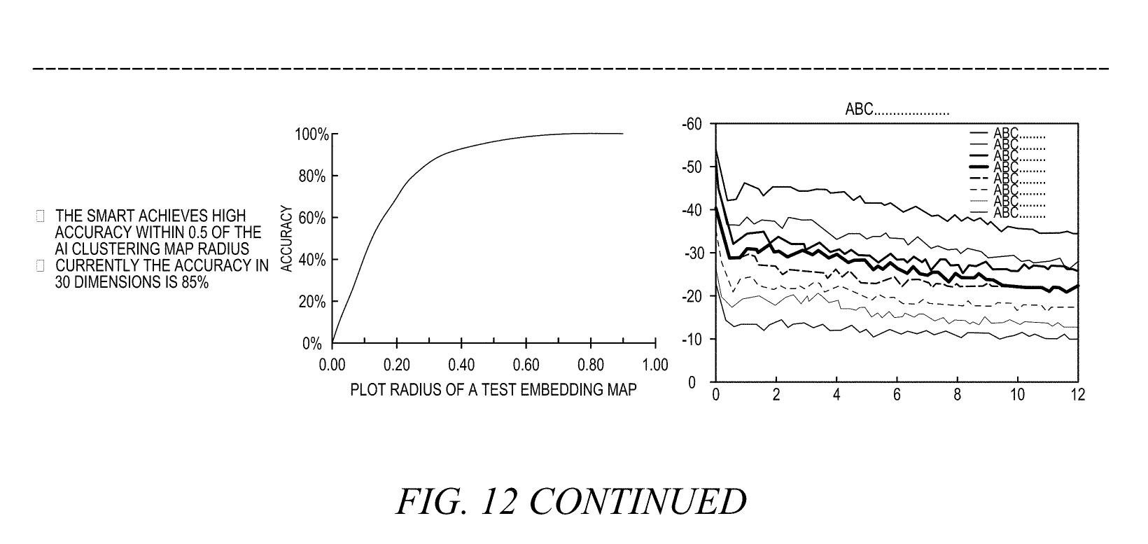

To explore the significance of cluster to cluster distance in the TAM, the types of structures present in three clusters was evaluated that were well defined and in varying proximity to one another. See boxes A, B and C of FIG. 12, which demonstrate in greater details the chemical structures of boxes within the cluster map 26, which shows an AI clustering map containing 2054 spectra, 83,000 iterations. A modified multilayer algorithm was used for the training method, and the results are visualized in AI clustering maps with nodes representing each spectra and inter-constellation distance for structural similarity. Similar molecules are mapped to nearby locations in the AI clustering map.

Cluster A was composed of oxidized steroids of highly similar structure to one another from plants Aphanamixis polystachya and Aphanamixis grandifolia whereas nearby cluster B was formed from a series of triterpene glycosides. The more distant cluster C contained several diterpenoids. The averaged 2D Tanimoto score (the 2D Tanimoto score (0-100) gives a quantification of structural similarity of two molecules) between compounds in the cluster A and B, T_{AB}=44, outbid the value T_{AC}=43 between compounds in the cluster A and C, which indicates that the DCNN method is better at quantifying and presenting structural differences among compound subfamilies than the algorithm used to generate 2D Tanimoto scores. Therefore, it is apparent that the SMART TAM not only recognizes closely similar compounds, but also appropriately places clusters of different compounds in their proper context relative to one another.

It is noted as an aside that the average intracluster Tanimoto score of the cluster containing aphanamixoids C, D, E, F and G is 95.7. The average intracluster Tanimoto score of the cluster containing uralsaponins A, B, C, F, M, T, V, W, X and Y is 96.3. The average intracluster Tanimoto score of the cluster containing ebractenoids A, B C, D, E, F, G, H, I and J is 69.4. All of these intracluster Tanimoto scores are higher than the inter-cluster Tanimoto score TAB=44 or TAC=43.

### SMART Characterization of Viequeamides of NRPS Origin

As a practical example of the functional use of the SMART workflow to discover new compounds, a group of marine cyclic depsipeptides was targeted, the viequeamides, in the context of efforts to study the anti-cancer activity of these marine cyanobacterial NPs. In this regard, a set of unknown compounds were isolated from the marine cyanobacteria 1) Rivularia sp. collected in Vieques, Puerto Rico and 2) Moorea producens collected in American Samoa, and then the same was purified by high performance liquid chromatography (HPLC). NUS ¹H ¹³C HSQC data were collected with 40% sampling density in the indirect dimension for seven purified compounds. Data reconstruction as described above for the seven samples yielded the visual HSQC spectra, and these were subjected to the SMART workflow to generate a TEM for their spectral inputs. The TEM embedded TAM was further interrogated, and it was found that the seven nodes during the test embedding clustered with nodes on the TAM for the previously discovered viequeamides A (1) and B (4). After an interplay of various 2D NMR spectra, the MS data, IR spectra and UV spectra, the planar structures of those seven compounds were determined. Stereochemistry of these new viequeamides was then elucidated by Marfey's analysis and/or X-ray crystallography, completing their structure determination.

Subsequent evaluation to H-460 human lung cancer cells revealed two of these viequeamides to have highly potent anti-cancer properties; viequeamide A2 (2) had an IC₅₀=0.62 μM and viequeamide A3 (3) had an IC₅₀=1.98 μM. Viequeamides B, C, D, G, H, and X showed no appreciable H-460 cytotoxicity.

Notably, the high resolution spectra obtained through the new 2D HSQC techniques discussed in the previous section can potentially raise the successful rate of deep learning assisted spectra profiling due to few artifacts in the spectra.

### EXAMPLES

### NUS 2D NMR Data Generation

A synthetic NMR standard (strychnine, 50 nmole TCI America, Catalog No. S0249) and several isolated NPs (viequeamides) were used in this study. The viequeamides were isolated from two different marine cyanobacteria; Rivularia sp. collected in Vieques, Puerto Rico and Moorea producens collected in American Samoa. 2D NMR spectra were recorded on a 600 MHz Bruker Avance III spectrometer with a 1.7 mm Bruker TXI MicroCryoProbe™. The solvent CDCl₃ contained 0.03% v/v trimethylsilane (δ_{H} 0.0 and δ_{C} 77.16 as internal standards using trimethylsilane and CDCl₃, respectively). All spectra were recorded with the sample temperature at 293 K.

Data were acquired using the NUS edited hsqcedetgpsisp2.3 HSQC pulse sequence. Data were acquired as 4096×32 points (32 out of 128 t₁ increments, 25% NUS) in direct and indirect dimensions, respectively, giving a total acquisition time of a quarter of its conventional counterpart. Spectral windows in direct and indirect dimensions were 7183.9 and 24118.9 Hz respectively. were processed by applying zero filling (round final size to power of 2) in both dimensions. Spectra were processed by applying IST with 400 iterations and forward-backward LP sequentially, or by applying IST with 400 iterations and MEM with the standard deviation of time-domain noise set to 200 sequentially. Data of viequeamides spectra were acquired and processed the same way, except that the indirect dimension were sampled with 40% NUS (102 out of 256 t₁ increments).

### Deep Convolutional Neural Network

### 1. Datasets and Processing

The dataset for HSQC spectra was compiled through collecting HSQC spectra from available online sources. Specifically, all usable ¹H-¹³C and ¹H-¹⁵N HSQC spectra (totally 4105), including cases of the same compound in different deuterated solvents, from the supporting information of Journal of Natural Products, years 2011, 2012, 2013, 2014 and 2015 were used in this analysis. In addition, the HSQC spectra of somocystinamide A and swinholide A in the supporting information of Organic Letters were also included in the dataset. All spectra were collected and initially processed by the following steps: (1) The HSQC spectra were saved as .png format images containing 600×600 pixels; (2) spectra rims, annotations, chemical structures, and other man-made marks were deleted using Photoshop such that only signal and noise were present in the images; (3) images were rotated and/or flipped when necessary to make sure that the direct dimension was ¹H NMR spectra with chemical shifts increasing from right to the left, and the indirect dimension was ¹³C NMR spectra with chemical shifts increasing from top to the bottom; (4) images were uniformly converted into black (signal and noise) and white (spectral background); (5) images from the same publication were pooled and labelled as the same training class. (6) since most of the images have unwanted “salt-and-pepper” noise, we applied a cross shaped 3×3 median filter before feeding to the neural. No other enhancements were applied.

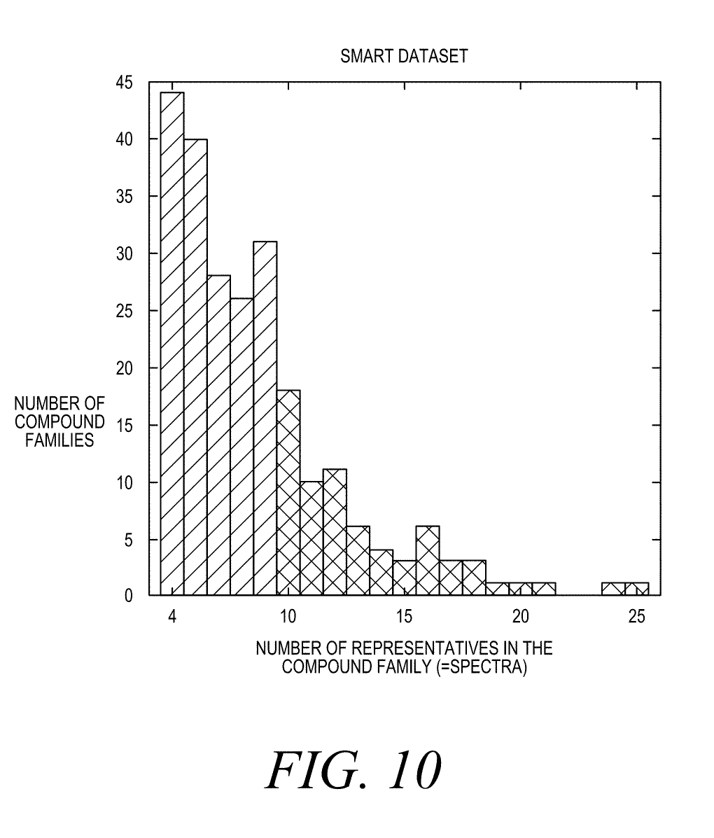

As seen in FIG. 10, which indicates the distribution of image number in each category of the training dataset, the dataset has a skewed distribution of images per class. In order to make training stable and comparison fair, classes were only selected that had at least 5 images. In total 238 categories (2054 images) were used, the largest having 25 and the lowest having 5 images per class. Thus, FIG. 10 illustrates a distribution in the training data set of numbers of families containing different numbers of individual compounds. The SMART5 training set contains 238 compound subfamilies, giving rise to 2,054 HSQC spectra in total, and this is indicated by all of the bars shown. The SMART10 training set contains 69 compound subfamilies and is composed of 911 HSQC spectra in total, and such is represented by the rightmost 14 bars (excluding the five leftmost bars).

A 10-fold validation scheme was used, randomly shuffling the dataset and splitting into the train, validation, and test set, in proportions 8:1:1. The procedure was repeated 10 times so that all images once become part of test set.

### 2. The Architecture of the DCNN

As depicted in table I below, which indicates the exemplary architecture of the DCNN used in this study, the DCNN architecture may include 9 layers with 4 convolutional layers, followed by 5 fully connected layers. Other numbers of various types of layers may also be used.

| Layer | | Number | | |
|---|---|---|---|---|
| Number | Layer Type | of Filters | Size | Additional Information |
| 1. | convolutional | 8 | 4 × 4 | maxpool 4 × 4 stride 2 |
| 2. | convolutional | 16 | 4 × 4 | maxpool 4 × 4 stride 2 |
| 3. | convolutional | 16 | 4 × 4 | maxpool 4 × 4 stride 2 |
| 4. | convolutional | 16 | 4 × 4 | maxpool 4 × 4 stride 2 |
| 5. | fully connected | — | 128 | dropout 0.5 |
| 6. | fully connected | — | 128 | dropout 0.5 |
| 7. | fully connected | — | 128 | dropout 0.5 |
| 8. | fully connected | | K | K dimensional embedding |
| | | | | layer |
| 9. | fully connected | | N_{c} | softmax |

An energy loss function was defined and applied to the outputs of the embedding layer (layer 8) and the cross-entropy terms applied to the softmax layer (layer 9).

To further avoid overfitting issues, dropout regularization was used. Dropout prevents parameter overfitting and drops out (zeros out) inputs at applied layers of the neural network with probability of 0.5 during training.

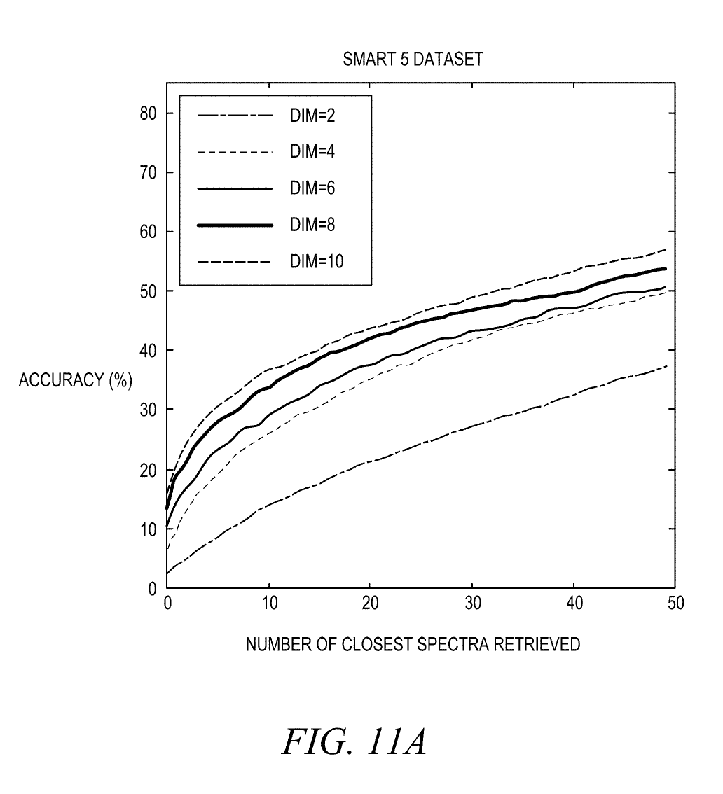

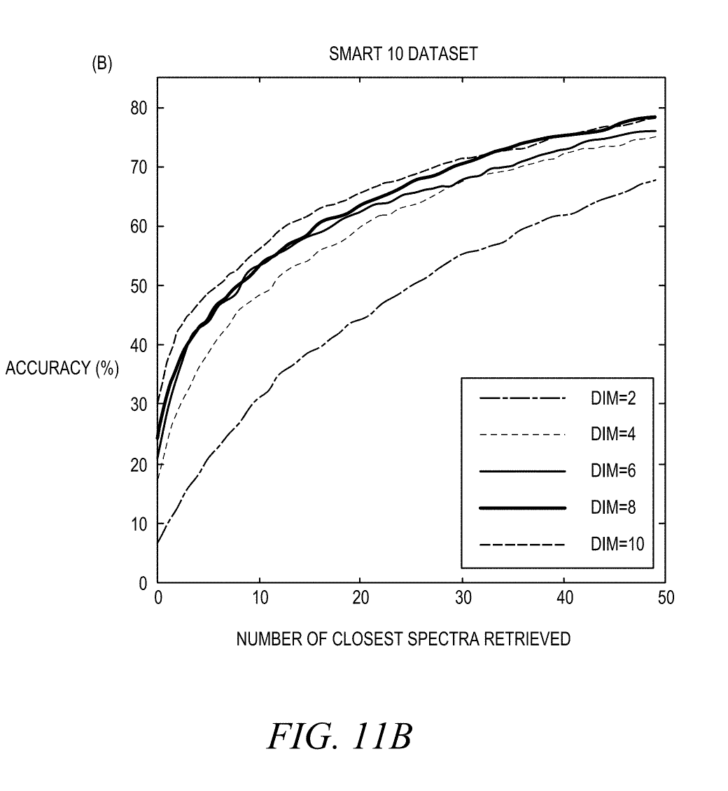

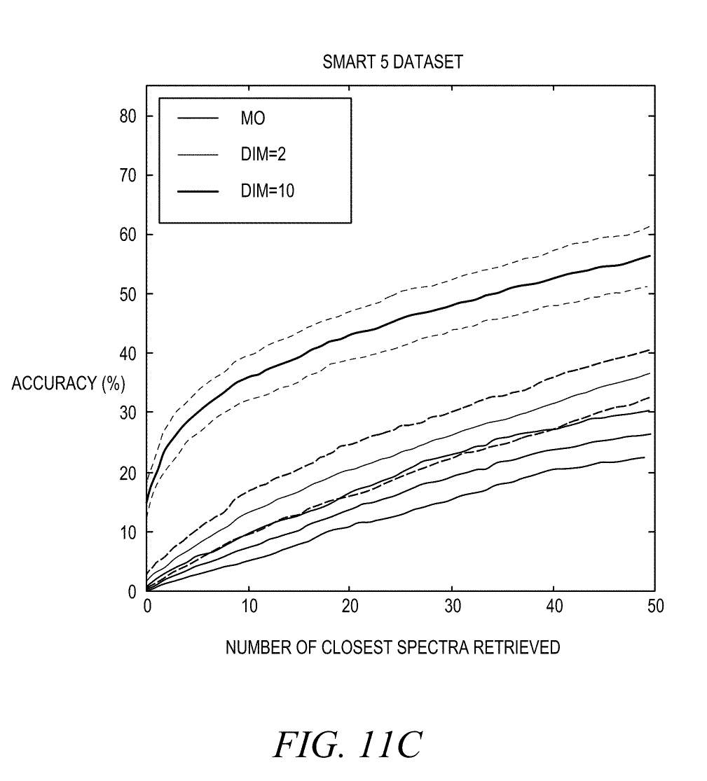

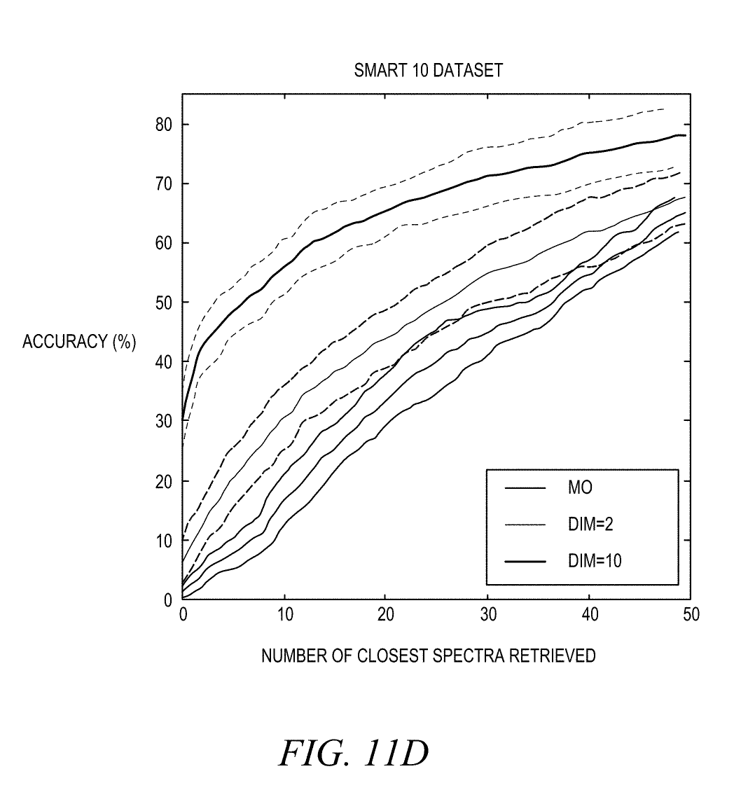

The dimensionality of the embedding layer affects performance in a straightforward manner. Several experiments were run to find the best K and to measure the accuracy on the validation set. Empirically, for given dataset, K=30 returns best results. See FIG. 11, which illustrates precision curves measured across 10 fold validation for different dimensions (dim) of embedding. For 11(a) and 11(b), mean precision curves on test sets are shown for SMART5 and SMART10 datasets, respectively. For 11(c) and 11(d), mean precision with error curves (dashed lines) for SMART5 and SMART10 is shown, respectively. In (c) and (d), the black plot (MO, most frequently occurring) is a baseline prediction of random compound associations on the basis of the number of members in a compound family. Specifically, the category with the most members is picked as the first compound association, the second most members as the second one, etc. This order is the same irrespective of the compound being considered.

With regard to the dimensions of embedding, it is noted that the Siamese network maps the compounds into a cluster space. The dimensions of that space are the dimensions of embedding. For example, if the Siamese network had two outputs, the compounds would be embedded into 2D. However, such was found to be somewhat restrictive, and thus 10 dimensions were employed, which appear to work well.

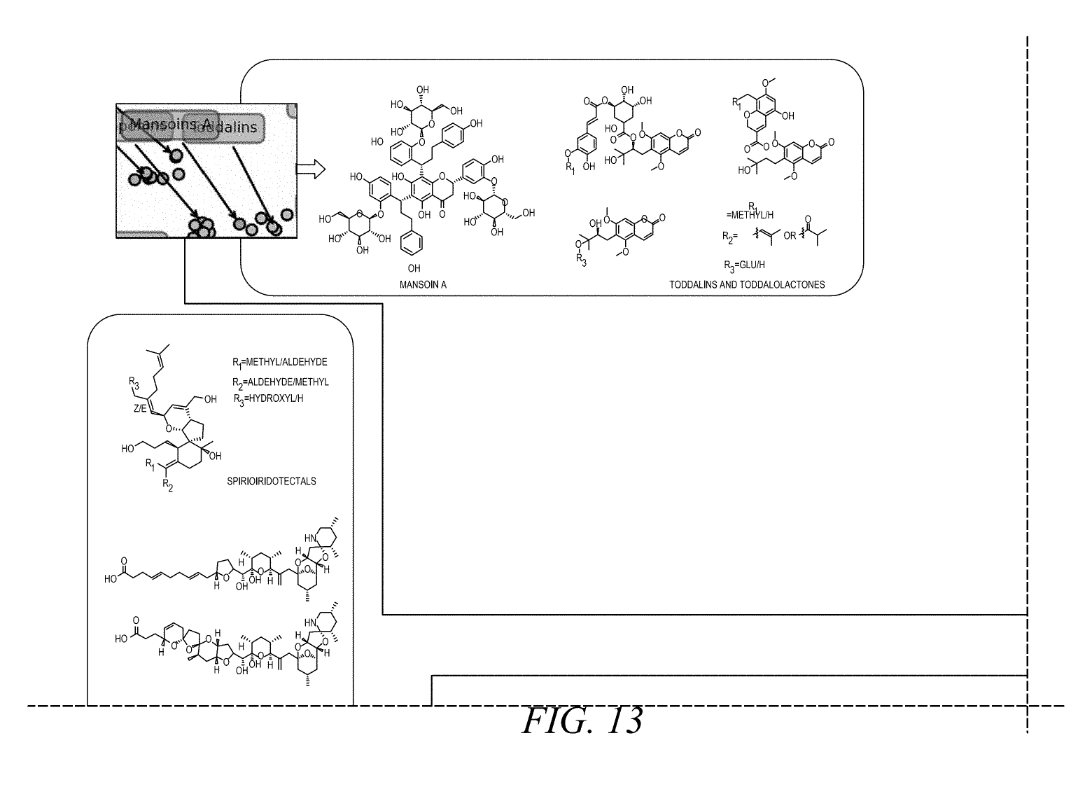

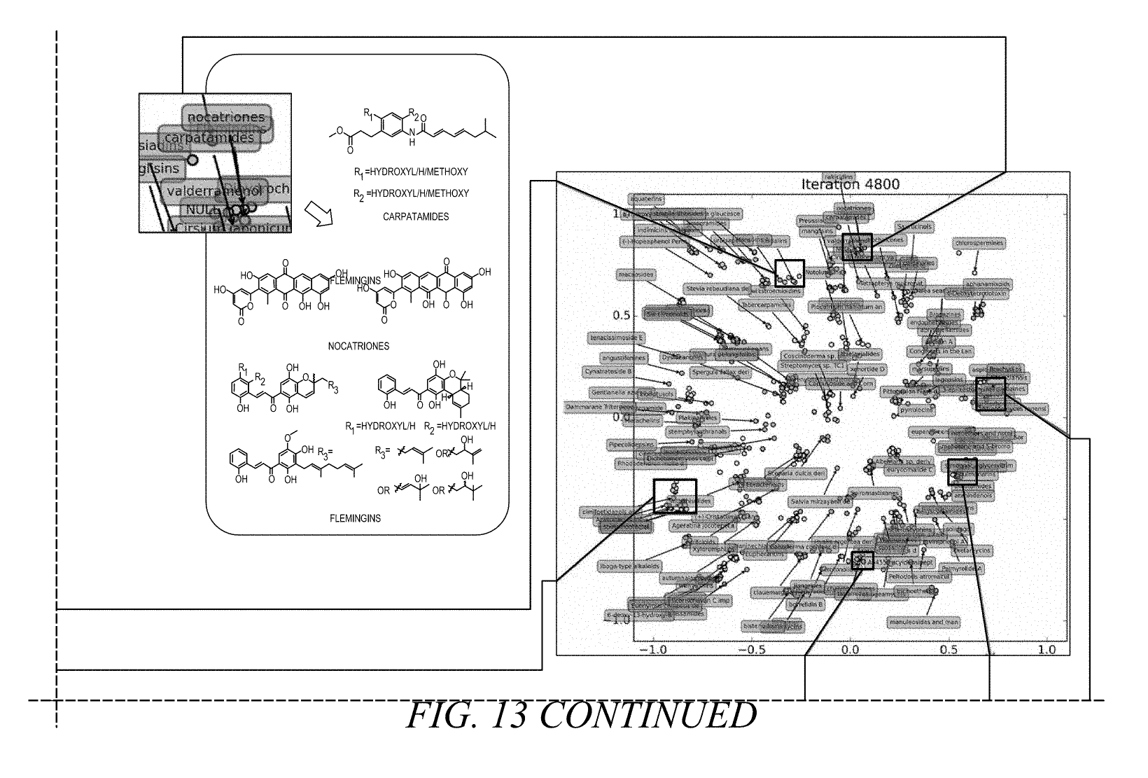

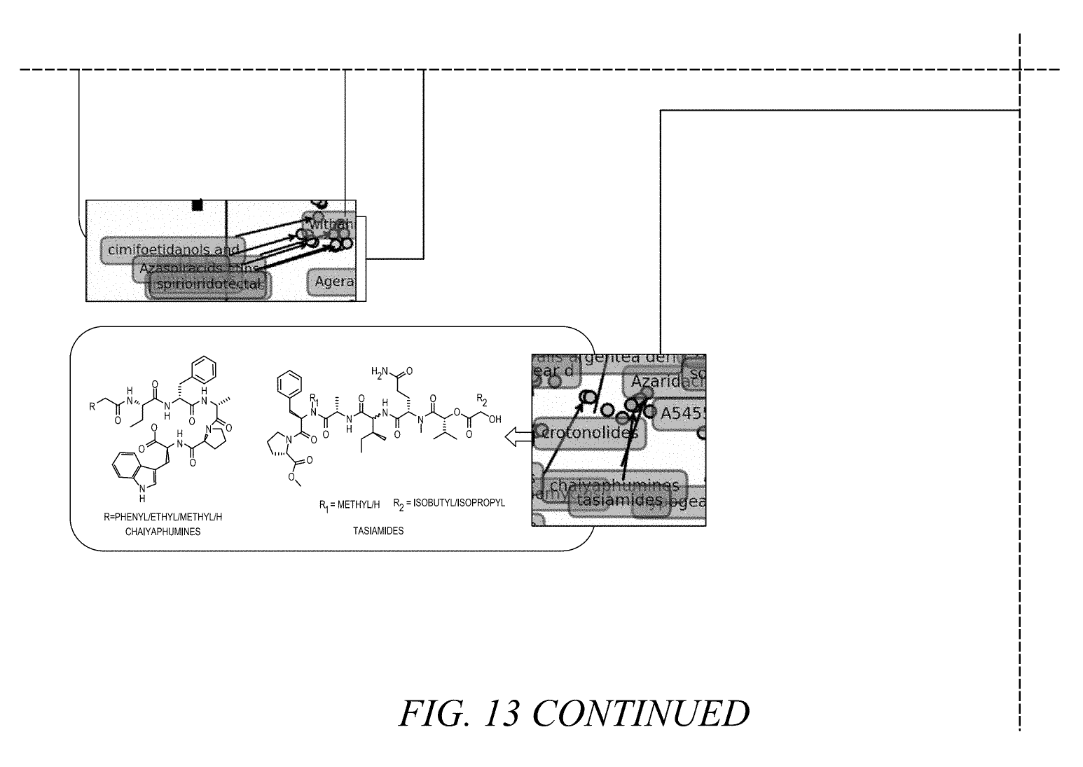

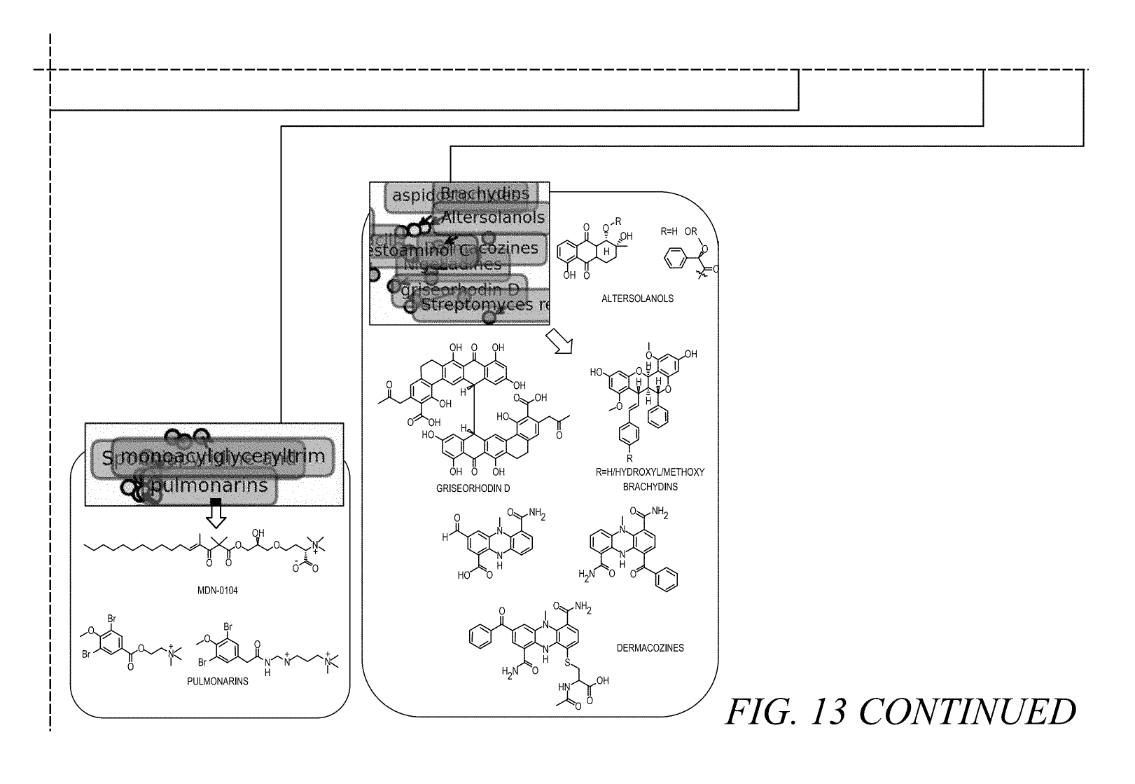

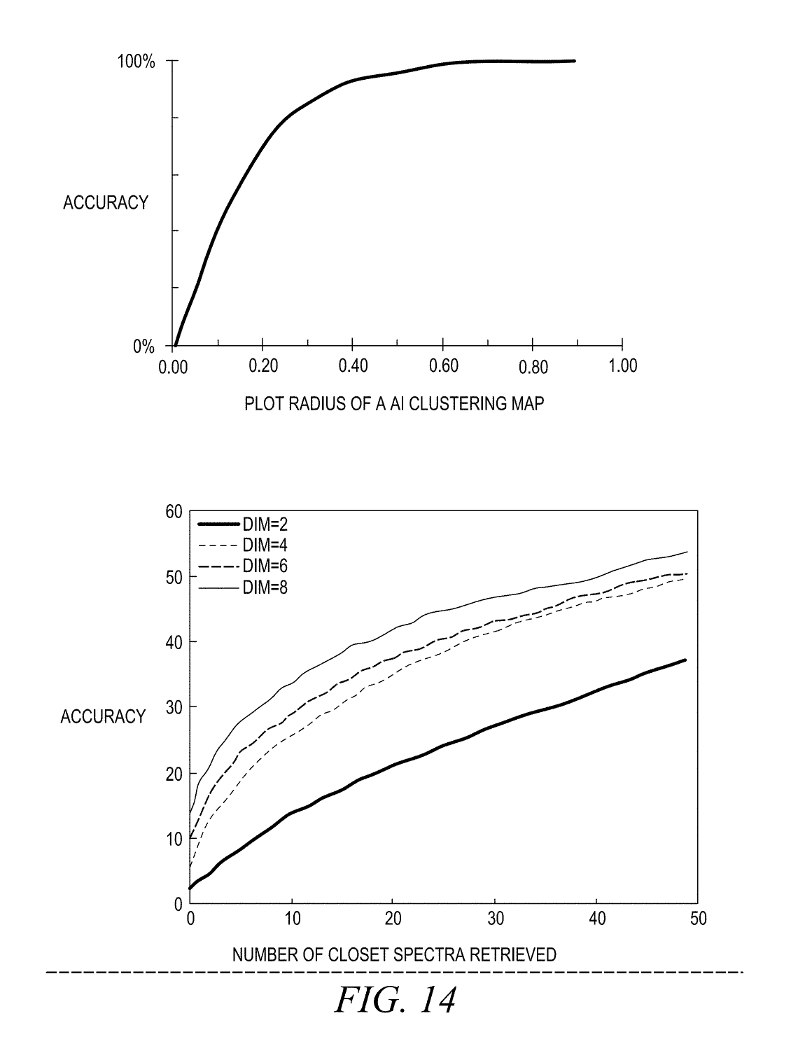

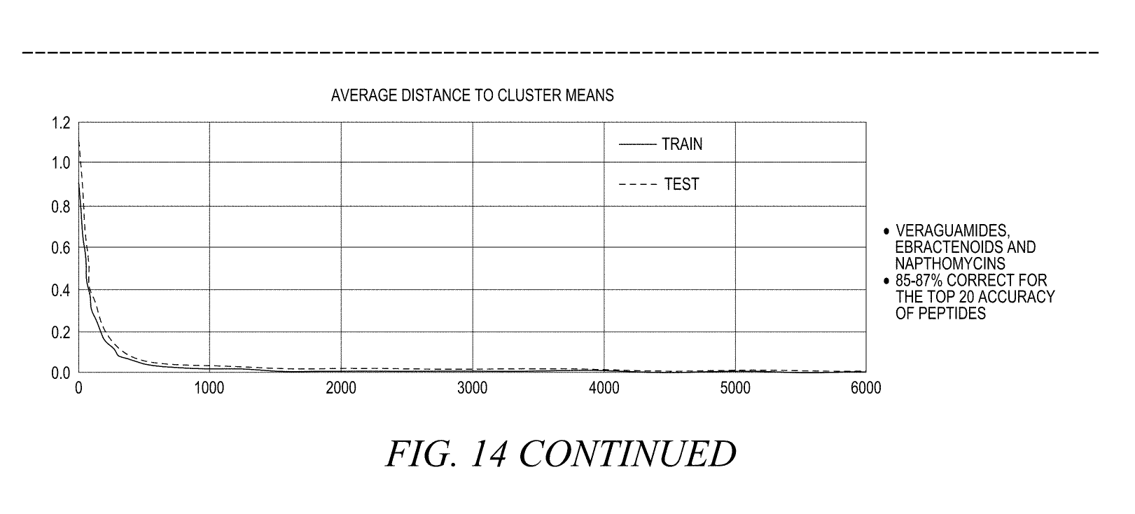

FIG. 13 indicates the results of 4800 iterations of training with only 400 compounds. As can be seen, a distribution of different families of compounds are depicted on the cluster map. And the change from one family to another seems continuous and evolving even when the data set is increased. From these results in others, it can be seen that spectra are embedded in the vicinity of their closest analogs in the AI clustering map. It is further noted, and referring to FIG. 13, that the training and use of the AI in this way has further increase the accuracy of the SMART tool. FIG. 14 indicates this increase in accuracy.

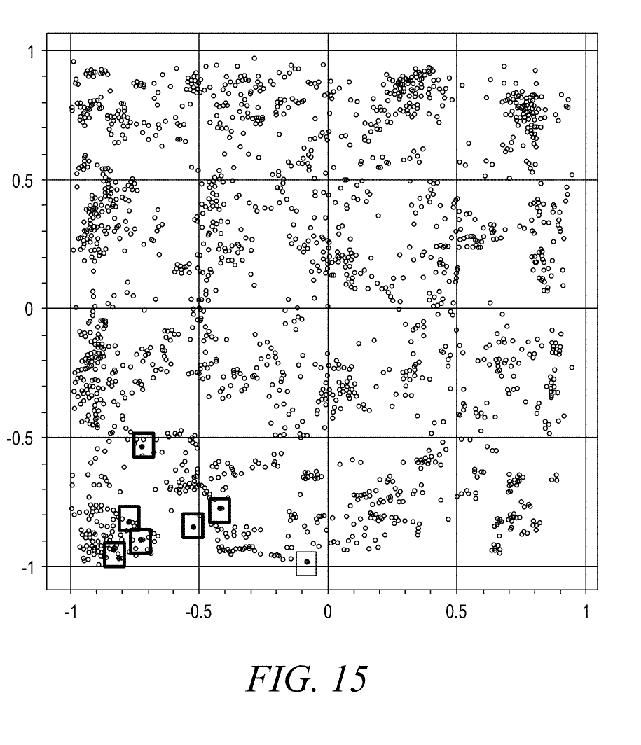

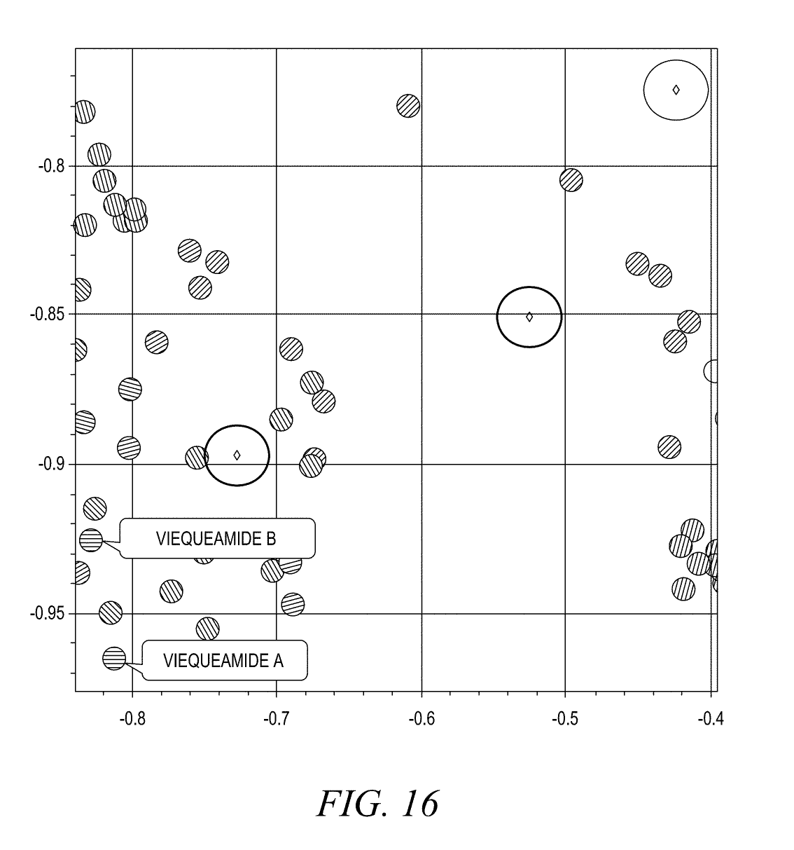

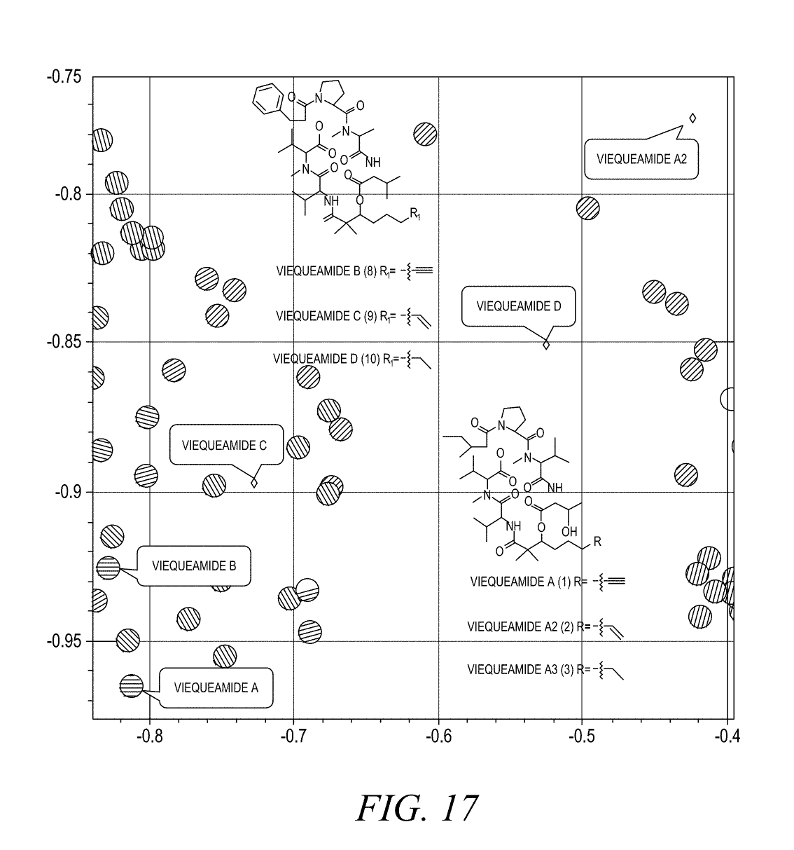

FIG. 15 illustrates results of embedding of unknown HSQC spectra into the clustering map. These are indicated by the red diamonds. The topmost red diamond is viqueamide A3, and the rightmost red diamond is viqueamide G. FIG. 16 illustrates an enlargement of the local area of the embedding map. The two orange nodes to the lower left are viequeamides A and B in the training set. Thus, newly isolated viequeamide A and B were de-replicated. FIG. 17 shows additional details.

As has been indicated above, the combination of 2D HSQC NMR and CNNs provide an especially useful combination. While fast, sparse, or NUS 2D NMR is not strictly required, 2D HSQC NMR and neural network learning, especially convolutional neural network learning, provide a highly useful combination with benefits that are not attainable otherwise. For example, deep learning allows creation of the most suitable set of features within the process of training, without any design or involvement by the investigator.

Moreover, CNN-based SMART performs better than conventional machine learning methods. Other approaches for automatic recognition of NMR spectra have appeared in the literature or private sector. The typical approach is to create grids over the data and then compute similarities based on how many points fall into the same grid cells. However, this approach can miss peaks that are near one another that happen to fall in different grid cells, so another approach is to use multiple grid resolutions and offsets before computing the similarities. Another method involves computer-aided structure elucidation (CASE, ACD/Labs) which is largely based on applying a least-squares regression (LSR) approach for comparing NMR chemical shifts; however, this tactic is not powerful enough to satisfactorily accommodate solvent effects, instrumental artifacts, or weak signal issues. An early effort using machine learning applied to NMR spectra was attempted which used Probabilistic Latent Semantic Indexing (PLSI), a method usually applied to text documents for information retrieval purposes. PLSI maps documents into a lower dimensional space using Singular Value Decomposition (SVD) applied to a document by word count matrix. To apply PLSI to compounds, the typical multi-scale and shifted grid cell approach was used, treating each grid cell as a “word” in the compound.

The aforementioned grid-cell approaches have certain similarities in that the shifted grid positions can be thought of as corresponding to the first layer of convolutions, which have small receptive fields (like grid cells), and they are shifted across the input space like shifted grids. The current approach also uses layers of convolutions that can capture multi-scale similarities. The grid-cell approaches, however, use hand-designed features, and the similarities are computed by simple distance measures. In particular, PLSI and LSR are linear techniques applied to hand-designed features. Furthermore, other representations, for example the ‘tree-based’ method, also rely on data structures designed by the researcher. The approach according to present principles, using deep networks and gradient descent, allows higher-level and nonlinear features to be learned in the service of the task. In particular, the CNN pattern recognition-based method can overcome solvent effects, instrumental artifacts, and weak signal issues.

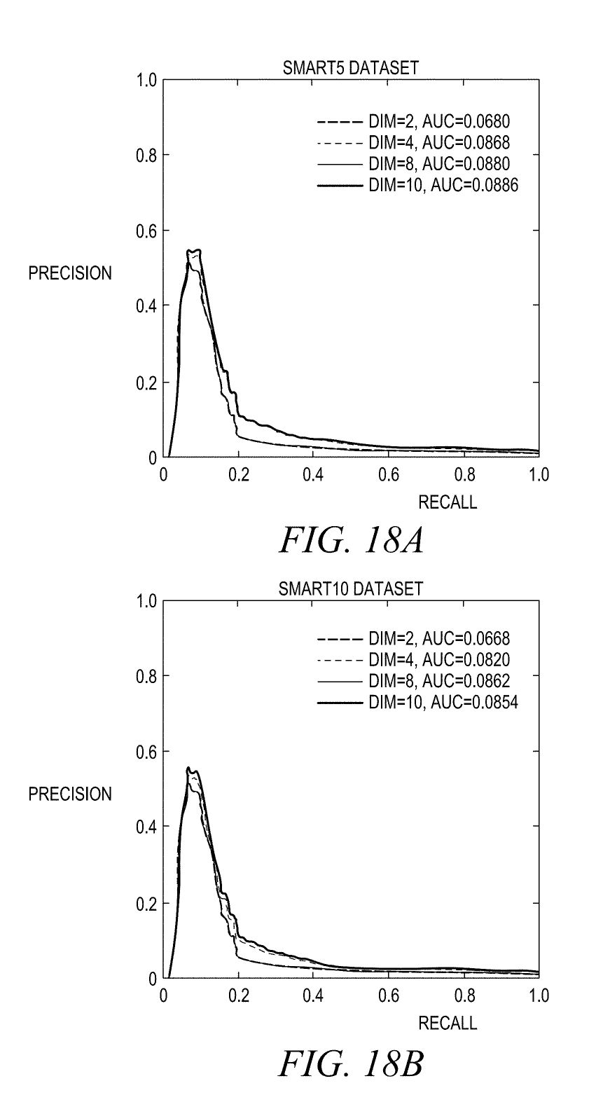

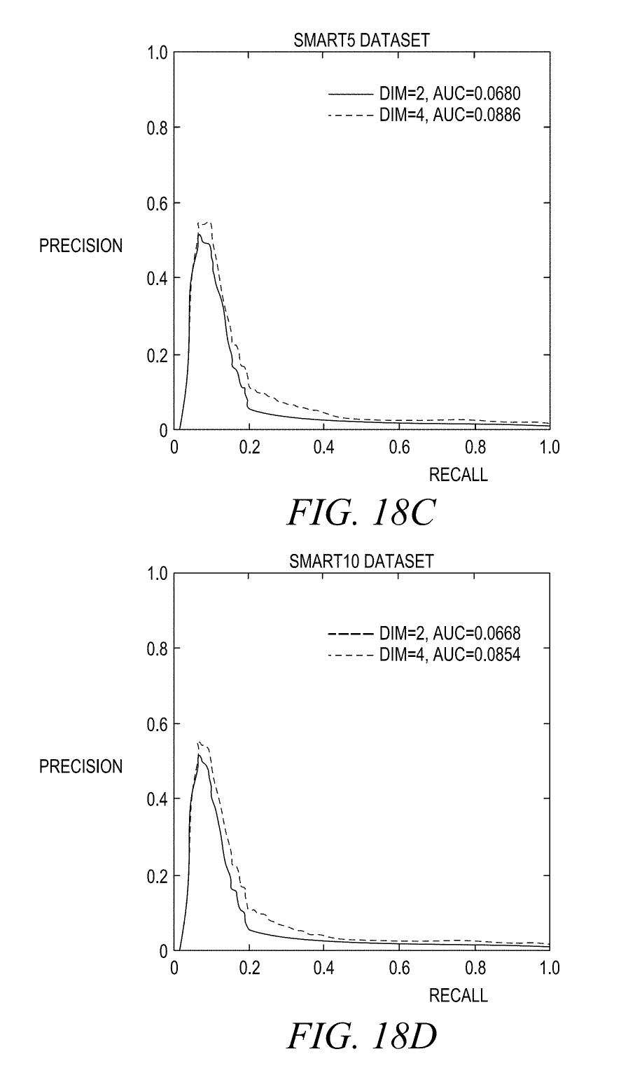

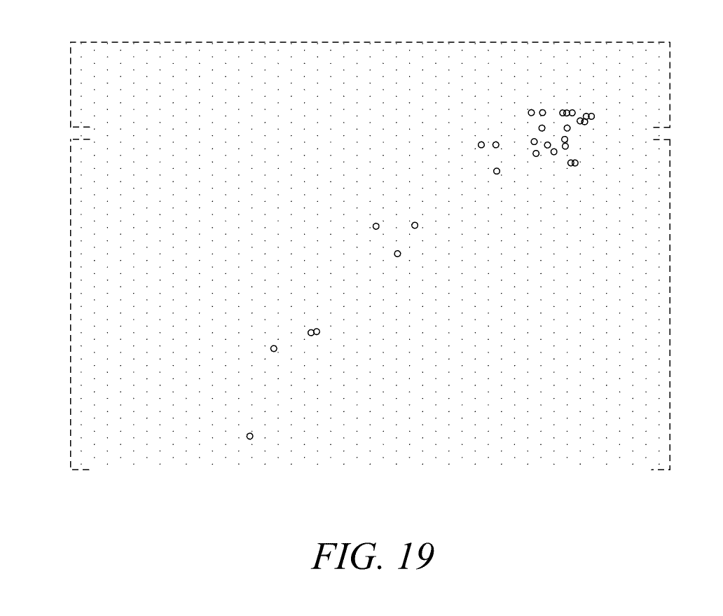

To further compare the instant approach with other NMR pattern recognition approaches, precision-recall curves were generated (FIG. 18) using the SMART trained with the SMART10 database. Regarding the SMART as a search engine, precision recall curves help evaluate the SMART's performance to find the most relevant chemical structures, while minimizing the non-relevant compounds that are retrieved. In our approach of HSQC spectra recognition/retrieval, precision is a measure of the percentage of correct compounds over the total number retrieved, while recall is the percentage of the total number of relevant compounds. Therefore, higher precision indicates a lower false positive rate, and higher recall indicates a lower false negative rate. The precision-recall curves of this approach show high precision peaks at low recall rates, suggesting that SMART retrieves at least some relevant structures in the first 10%-20% of compounds retrieved, and thus indicates that the SMART returns accurate chemical structures and finds a majority of all positive similar structures. It is noted that these curves are not comparable to the results reported above using PLSI, as those researchers had a much smaller number of compounds overall, making it easier to retrieve relevant compounds.

The precision recall curves (FIG. 18) of SMART were recorded by randomly selecting HSQC spectra from the test dataset and recalling closely related HSQC spectra from the training set. In this regard, precision was calculated by dividing the number of true positives over the combination of the number of true positives and the number of false positives. Recall was derived by dividing the number of true positives over the combination of the number of true positives plus the number of false negatives. The F1 score was derived as the harmonic mean of precision and recall. In this experiment, for each compound in the test dataset, a precision recall curve was calculated by calculating precision (the number of retrieved compounds that are relevant) and recall (the number of relevant compounds that are retrieved) of the retrieved HSQC spectra from the training dataset within an expanding hypersphere centered at the compound in the test dataset. These final precision recall curves were averaged over the test dataset. The CNN that was used in this regard were trained for 10,000 iterations on SMART5 and SMART10 dataset with 10-fold cross validation for embedding dimensions k=2, 4, 8, and 10.

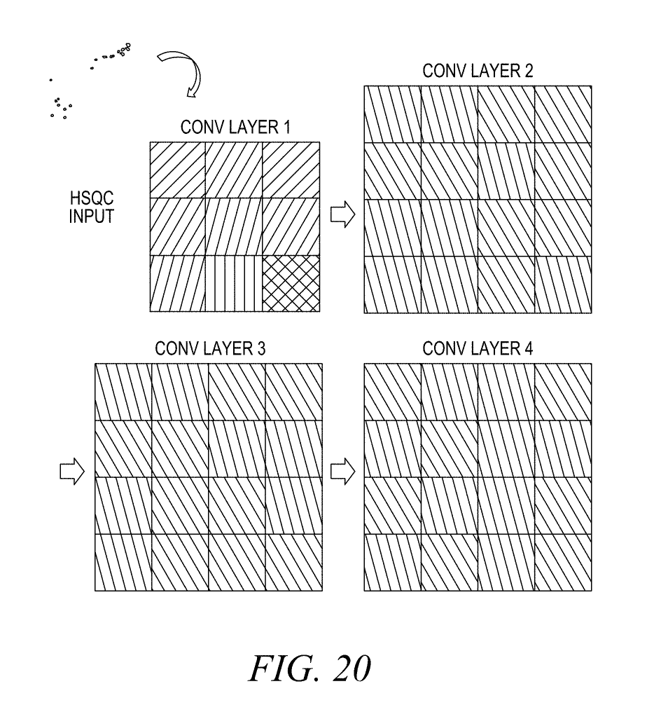

The features that are identified and used within the CNN are extracted by a deep network. Qualitatively, the features learned at the input level, which are based on the pixels, are fed into the next layer up, which computes nonlinear features of those features, and the third layer computes nonlinear features of nonlinear features of the first layer of linear features, etc. In one implementation, the “Tensorflow” package was employed to visualize the features that were learned by the different layers of the CNN, and the results show that the features become more abstract as the layers of the network are traversed. Visualizations of these features are seen in FIG. 20. In this figure, feature maps are extracted from convolutional layers 1, 2, 3 and 4 in Table I, respectively.

Regarding particular frameworks for performing deep learning, there exist a number of these, including Torch, Tensorflow, Theano, Caffe, mxnet, etc. There is no particular advantage of one over another, except for the syntax of the usage. In one implementation, Theano provided a version of a deep learning framework that had a) auto-gradient computation b) a good (native) python interface c) a stable development version.

The choice of parameters and hyperparameters completely empirical in the entire deep learning field, with no best method except trial and error. The reported parameters led to good results in all experiments. When searching for hyperparameters, GPU utilization/runtime per iteration were considered. There is a tradeoff between batch size, number of iterations of learning, and wall-clock time. GPUs are very good at processing batches of examples for training, and the more memory a GPU has, the larger batch it can process (higher utilization), which will reduce the noise in Stochastic gradient descent (SGD). However, the larger the batch, the more time it requires, but this in turn reduces the number of SGD iterations that are required. Another essential parameter to tune is the learning rate; if the learning rate is too high, the optimization procedure can diverge, whereas smaller rates may terminate before reaching the best minimum of the objective function. Based on preliminary experiments, a batch size of 200 pairs was chosen and an initial learning rate of 0.002. Other parameters were likewise chosen based on preliminary experiments, for example: margin=0.02, 12 regularization multiplier on dense layers: 0.0001, update rule=adagrad.

### 3. Loss Function Design

To enable the SMART to perform both qualitative decisions and quantitative measurements for NPs, the EBM was applied to associate a scalar energy to each configuration of the variables. A model was defined with two sets of variables, in this case, X and Y. X was denoted as a matrix containing the features/pixels from a 2D spectrum for an analogue in a compound family category. Variable Y is a discrete variable that represents the category of the analogue encoded. For example, if there are labels: curacins, apratoxins, lyngbyabellins, the label curacins would be encoded as [1, 0, 0, 0] and apratoxins as [1, 0, 0, 0] respectively. For each image Xᵢ there is a respective label Yᵢ.

In order to fully specify the method, the neural network graph may be denoted as G_{W}, where W is the weights of the neural network. The output of the neural network after layer l for image X is G_{W}^{l}(X). Moreover, the following distance function d may be defined between images Xᵢ and Xⱼ:

d(Xᵢ,Xⱼ)=∥G_{W}^{e}(Xᵢ)−G_{W}^{e}(Xⱼ)∥

where ∥⋅∥ is second norm of vector, and e is the index of embedding layer.

The training process searches for the optimal configuration of image embeddings that make the compounds within the same group be located close to each other. In order to facilitate this objective, the contrastive loss function may be defined following:

$$L ( {X}_{i} , {X}_{j} ) = \{ \begin{matrix}{d}^{2} ( {X}_{i} , {X}_{j} ) , if {Y}_{i} = {Y}_{j} \\ {max ( 0 , m - d ( {X}_{i} , {X}_{j} ) )}^{2} , otherwise\end{matrix}$$

where m is a hyperparameter that defines the margin. This loss function penalizes large distances between pairs of similar images (first line), but for the dissimilar images penalization only occurs if images are within m units. This loss function ensures that images would form well behaved clusters during embedding.

During the course of the experiments, locations of cluster means was observed to change drastically. In order to avert this, a softmax layer was defined following the embedding layer, and forced the neural network to predict the correct labels. Thus, the final loss function is:

\hat{L}(Xᵢ,Xⱼ)=L(Xᵢ,Xⱼ)+H(Ŷₗ,Yᵢ)+H(Ŷⱼ,Yⱼ)

where Ŷ is output of neural neural network after softmax layer for image X (e.g. G_{W}^{s}(X)), and H is the cross-entropy function.

In conclusion, what has been described is SMART, which is the first ensemble of 2D NMR and a convolutional neural network such as DCNN. This tool streamlines dereplication and determination of NPs from multiple organisms and facilitates their isolation, structural elucidation and biological and ecological evaluation, which leads to an increased appreciation for the structural diversity and theranostic potential of NPs. Using systems and methods according to present principles, what may be envisioned is a future of reshaped NPR in which NP researchers use SMART to support structure dereplication and assignment to molecular structure families, and thus, augment their research capacity. With further refinement of the SMART workflow such as training for spectra of the same compound with different S/N ratio, deeper understanding of S/N ratio on spectral recognition and marrying to other fast NMR techniques, SMART possesses the potential to create opportunities in other natural products research directions.

Systems and methods according to present principles may be employed in a number of fields, including: structural elucidation of complex chemical compounds; general chemical industries; drug discovery and development environmental monitoring (chemical signaling, quorem sensing, etc.); clinical diagnosis and metabolomics; quality control of mixtures; nuclear magnetic resonance software industry; chemical biology, chemical ecology, drug discovery and development, pharmacology and the total chemical synthesis of NPs. Precision recall curves may be employed that show that systems and methods according to present principles may be employed as a search engine, to find the most relevant chemical structures. For example, the systems and methods disclosed can determine if retrieved compounds are truly relevant, as well as determining if all relevant compounds found in a search have been retrieved. In particular, and referring to FIG. 18, precision recall curves are illustrated, where the “precision” indicate the percentage of correct compounds over the total number retrieved, and the “recall” indicates the percentage of the total number of relevant compounds.

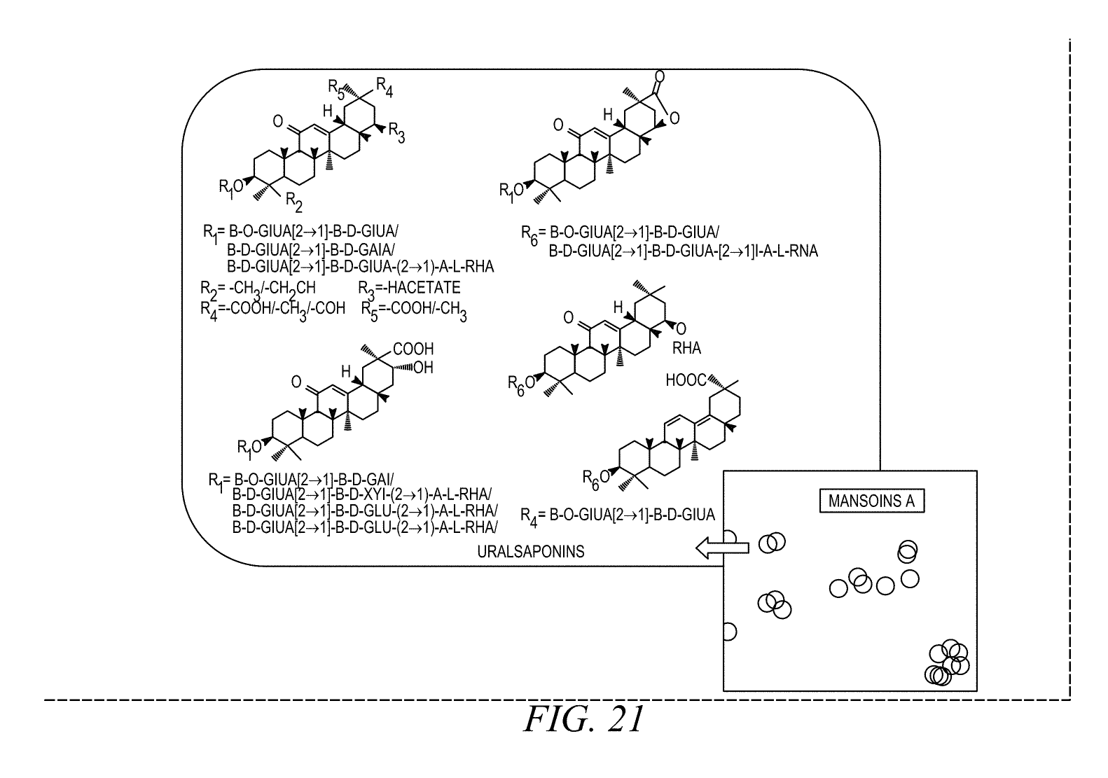

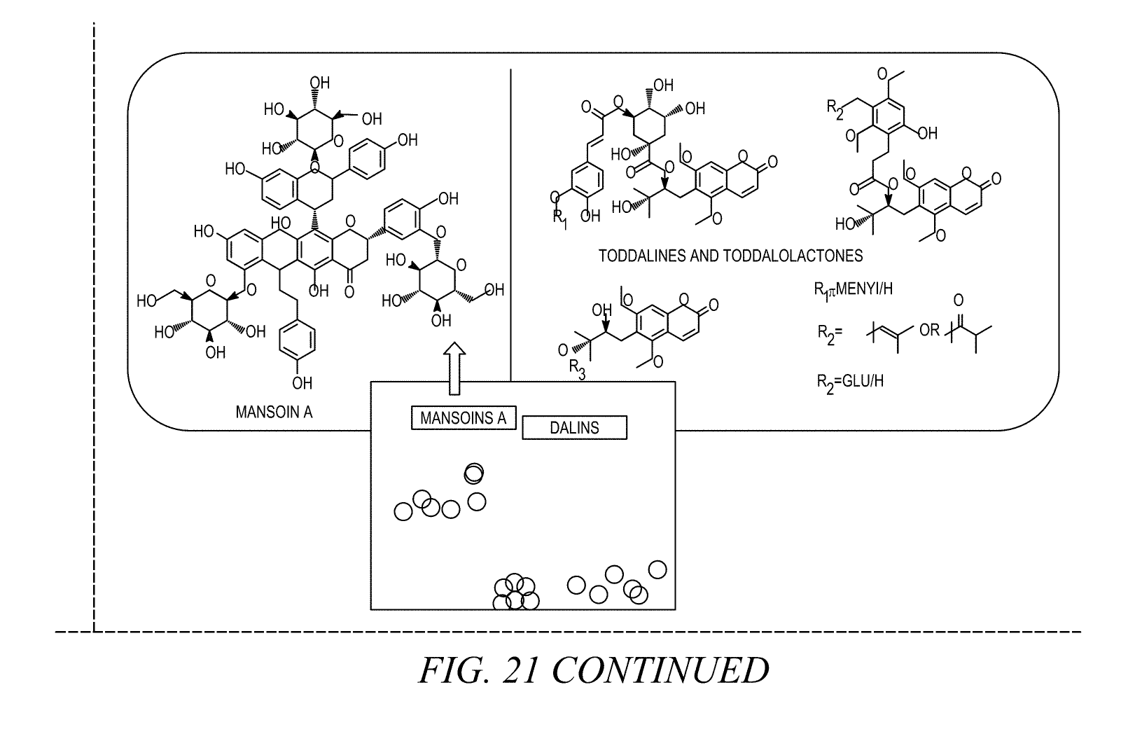

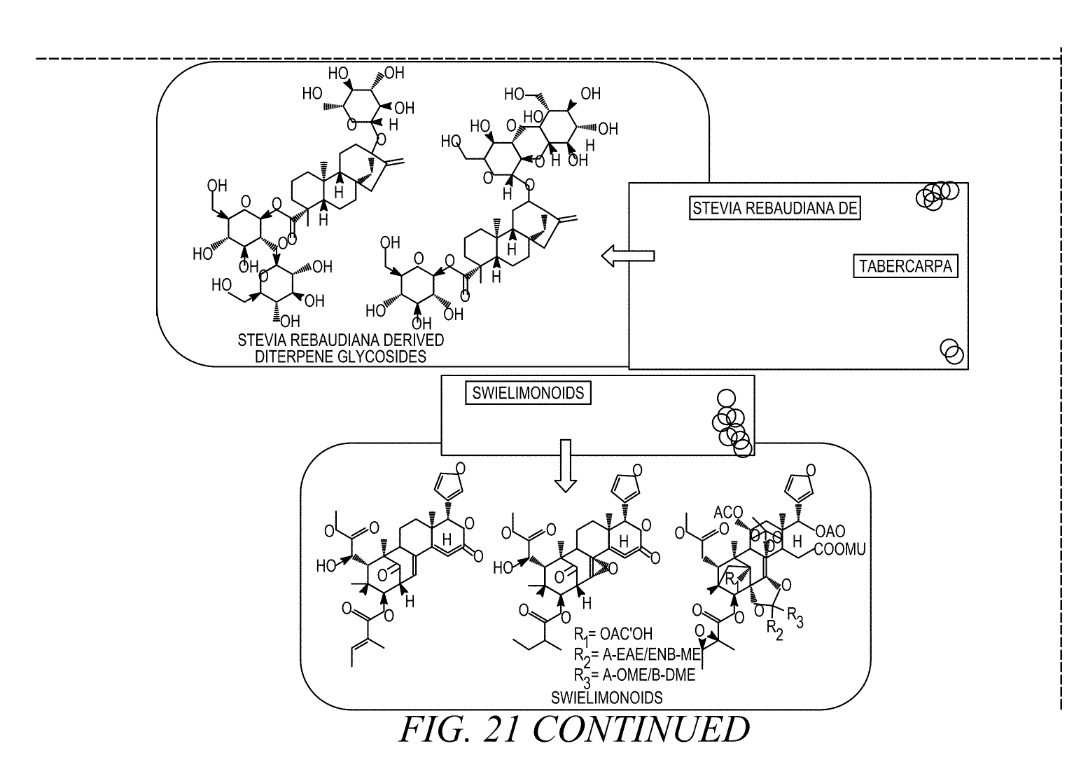

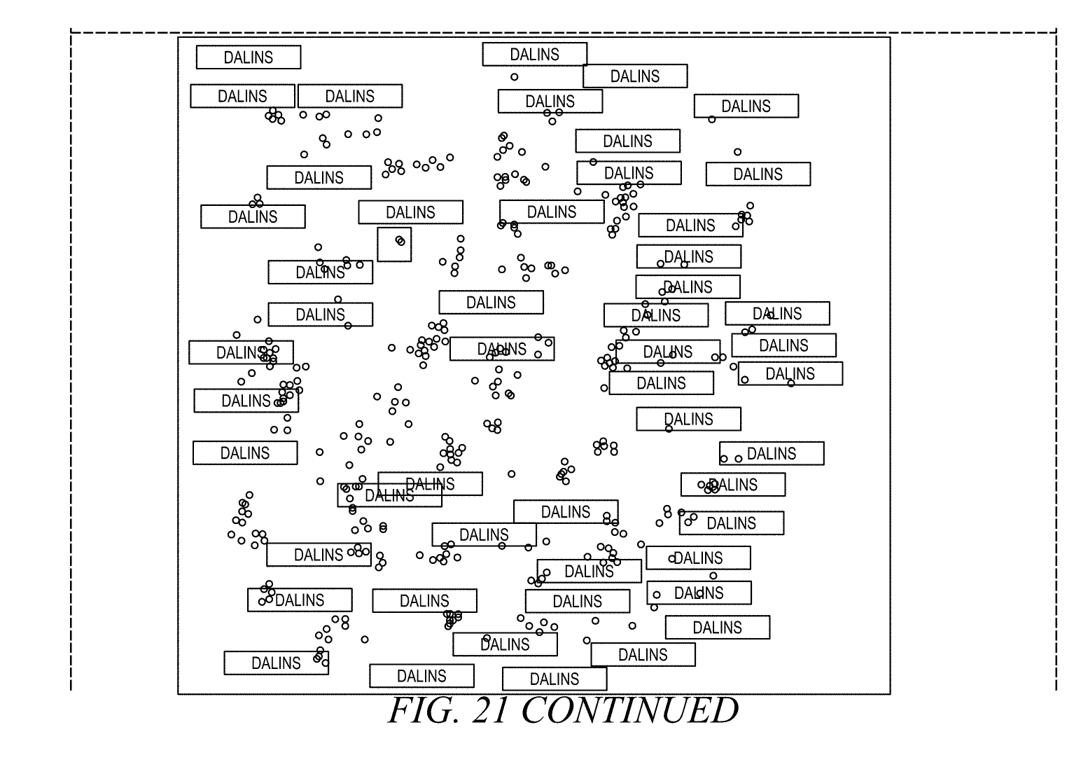

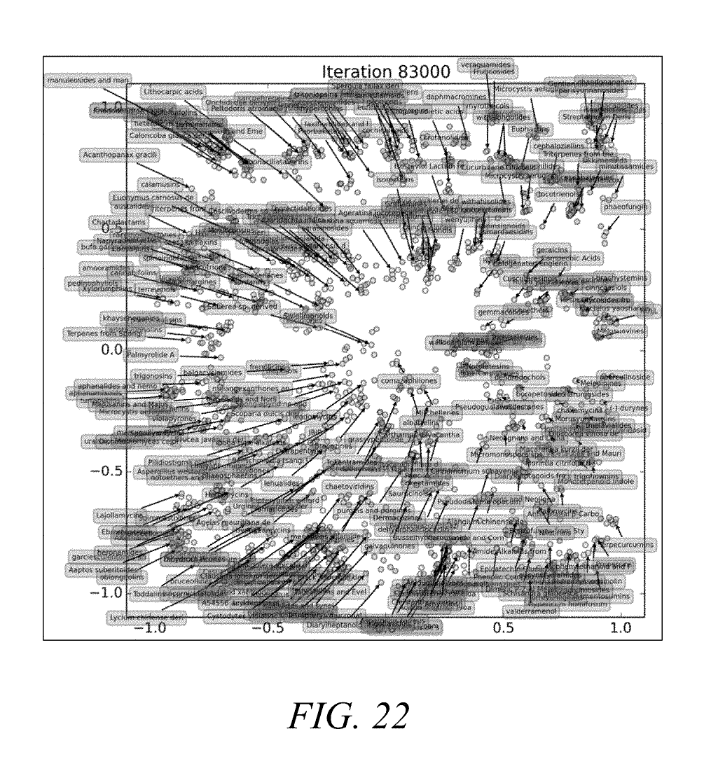

FIG. 21 illustrates how the distribution of molecular families on the clustering map evolves over time and with continuing iterations. For example, FIG. 22 shows a 83000 iteration training result.

In one variation, and to indicate the robustness of the systems and methods disclosed, both experimental and “fake” 2D HSQC spectra may be used for training the convolutional neural network. In one exemplary method, noise was added to each experimental spectrum, mimicking the white noise generated during real experiments. In another method, the signal area within a given experimental spectra was deleted—this type of artificial HSQC spectra is needed in order to demonstrate tolerance of small differences among compounds within the same compound family. A third method was employed that, randomly, moved a number of the existing signals to new locations within a radius of the geometric center of the signal. This type of artificial HSQC spectra is designed but not limited to tolerate solvent effects of the same molecules. In one experiment, white noise was added to each increment of the indirect dimension of a 2D NMR spectra. A 2D Fast Fourier Transform was applied to this noise matrix. The intensity of the added noise was increased consecutively in a finite arithmetic progression of 100 times, rendering 100 noise maps for each spectrum. Despite the subsequent and significant increase in noise, systems and methods according to present principles were still able to discriminate the innate spectra. This shows that the systems and methods can be trained to read HSQC signal patterns while ignoring less informative white noise.

In more detail, in order to quantify the robustness, two HSQC spectra in the SMART10 database were randomly selected, namely, “hyphenrone I” and “ebractenoid C”, and white noise was applied to these two spectra by simulating the 2D HSQC NMR experimental white noise. Specifically, a 2D matrix of white Gaussian noise was added to each increment of the indirect dimension of a 2D NMR experiment that contained no other signals. The column represents the time delay between two HSQC pulse sequences, while the row represents the free induction decay collecting time for each scan. Then, 2D Fast Fourier Transformation was applied to this noise matrix to give rise to simulated noise peaks on the 2D NMR output. This simulated noise was then separately added to the HSQC spectra of hyphenrone I and ebractenoid C using Matlab 2013. The intensity of the added noise was increased consecutively in a finite arithmetic progression of 100 times, rendering 100 noise maps for each spectrum. Those noise maps as well as their original spectra were exposed to the convolutional neural networks pretrained with SMART10 for over 10,000 iterations.

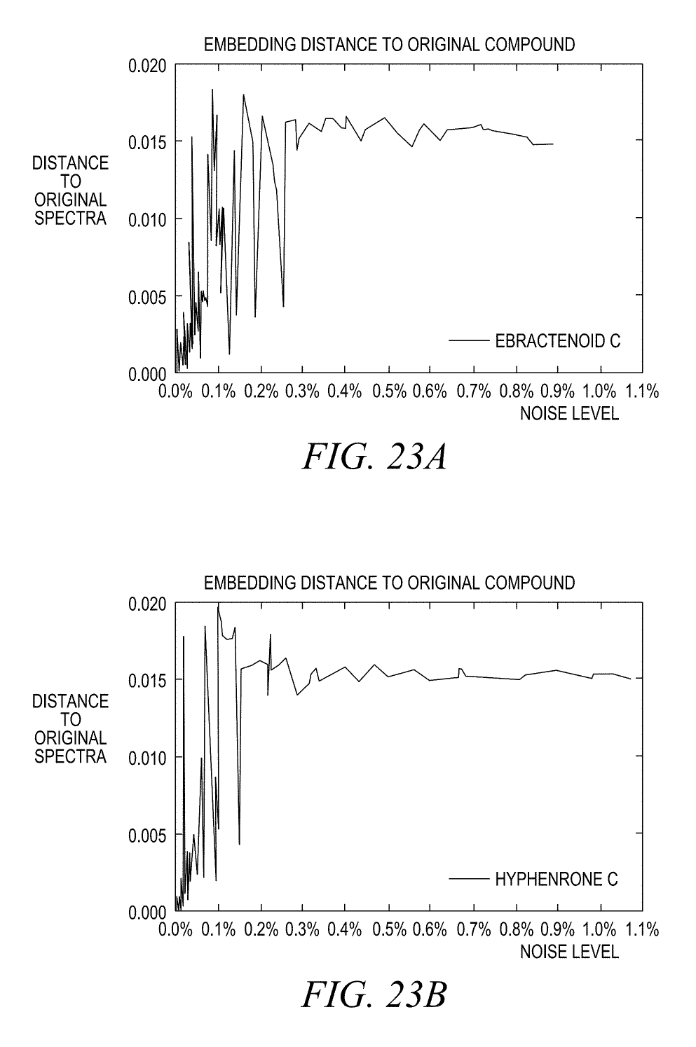

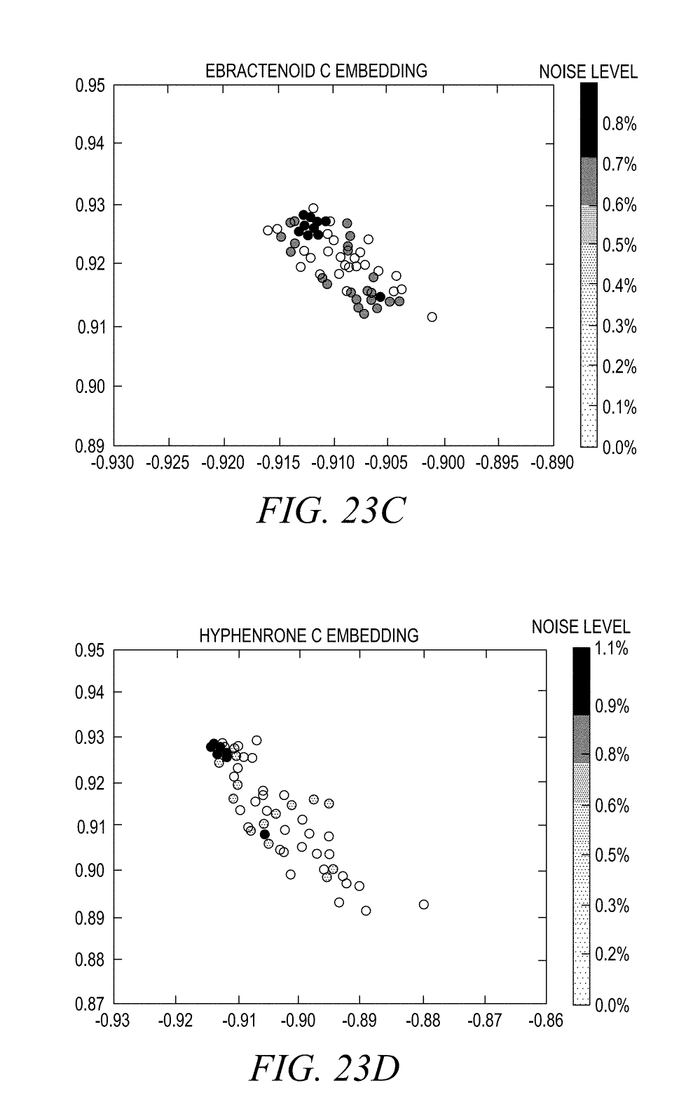

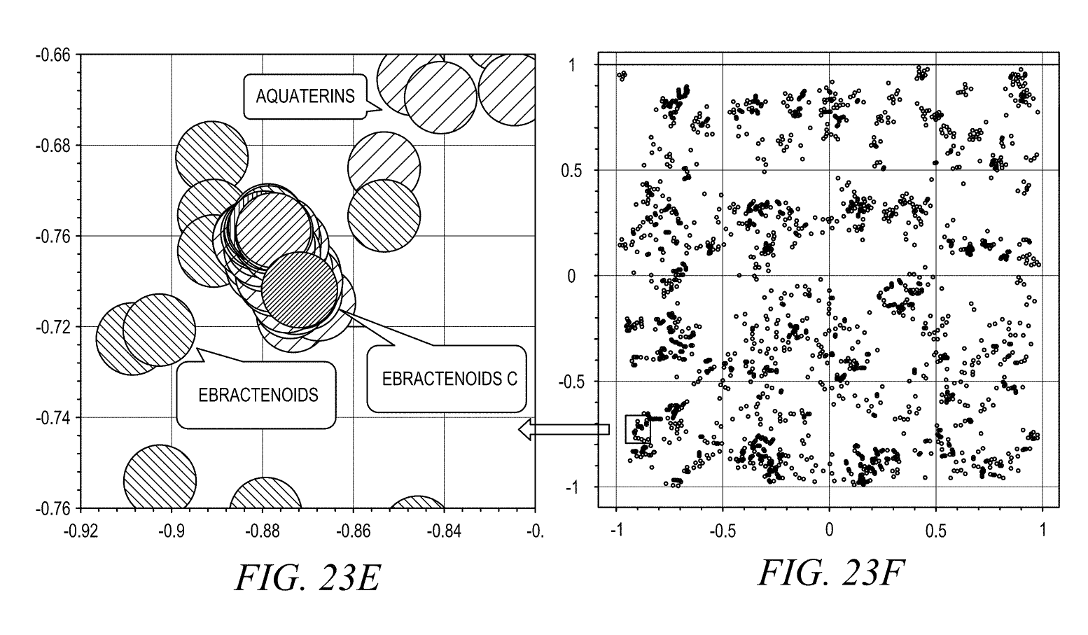

The results were shown as two distance vs. noise power plots shown in FIGS. 23A-23D. The distance measured in the y-axis of these two plots was in the same non-physical unit as the clustering maps above. The results were also visualized in 2D clustering maps with each node representing one noised spectra, and intensified node color correlating with increased noise level. See also FIGS. 23C and 23D. The original image without added noise is shown as the black node in those 3D clustering maps.

In more detail, FIG. 23A-23F illustrates distance of the noised spectra measured against the original spectra of ebracenoid C and hyphenrone I. In FIGS. 23A and 23B, the distance measured in the y axis of these two plots was in the same non-physical unit as the clustering maps described above. The noise level is defined by percentage of pixels altered to noise versus the total number of pixels of the HSQC spectra. In FIGS. 23C and 23D, the results were also visualized in 2D clustering maps with each node representing one noised spectra, and intensified node color correlating with increased noise level. The original image without added noise is shown as the black node in those 2D clustering maps. The nodes in 23C were then embedded to a global view of 2D clustering map in 23F, and provided a marquee zoom view (23E) of the area in the red box of (23F). FIG. 23E shows that noised HSQC spectra are clustered close to their original spectra, and thus does not confuse chemists with other nodes representing the HSQC spectra within the same compound subfamily (ebractenoids in this case).

In other words, SMART is robust for noised HSQC spectra. Even with a low signal to noise ratio, the SMART still recognized the spectra. By adding noise to HSQC spectra in the SMART10 database and measuring the matric distance of those noised spectra to their original ones, it was observed that when noise intensity increases, the distance also increases. However, the noised spectra were still effectively recognized as being closely related to their original compounds.

Systems and methods may be employed for novel compound discovery, building a user-friendly web service platform to facilitate HSQC and other data cure ration, and making a clustering map in virtual-reality devices, and so on. To further supplement the data, collection locations and bioactivity information may be added. Edited gradients selected to the HSQC spectra may be integrated into the data set as well. In addition, deep learning may be employed and applied with the compound structures themselves, as images, and then results may be compared between the maps created with the structures versus the HSQC spectra.

It is noted that the cluster maps described have been shown as 2D maps. However, the same generally constitute a collapsing of an N-degree representation, depending on the number of dimensions in a vector representing points in its respective spectra. The maps may also be visualized in 3D, however, and 3D representations may provide even additional details, elucidating structural similarities where the same are not seen or are hidden in 2D representations.

Systems and methods according to present principles vastly speed up the process of natural products discovery. Currently, natural products discovery is a very slow process. However, in order to find new drugs of significant bioactivity, currently natural products discovery is a step that cannot be skipped. Natural products are secondary metabolites produced by terrestrial and marine species that have molecular structures that are so ingenious that they cannot be created by human beings, and the same work against disease and/or can benefit human beings in many ways. The workflow of natural products discovery is as follows. Pure compounds are isolated from field collected species, and then there chemical structures are determined using 2D NMR techniques. Only after the structure determination is finished can the pure compounds be tested bioactivity. The order of this process cannot be changed and no steps can be skipped. Because a natural product requires significant testing, e.g., approval by the Food and Drug Administration, before use by human beings, it must show clearly its chemical structure. For a researcher, once he/she tests the pure compound for bioactivity, the compound cannot be recovered. So the 2D NMR based structural determination is the bottleneck. There are two reasons for this. One is that 2D NMR experimental time is slow. The conventional software that drives an NMR machine for the 2D NMR experiment is very time consuming, especially when the pure natural product sample is scarce. The second reason is that the 2D NMR spectra analysis by researchers is time consuming. The average time is three weeks to half a year for simple chemical compounds. For the first challenge, systems and methods according to present principles employ a Fast NMR technique, which improves the efficiency of the NMR machine by cutting time to a quarter or ⅛ or the original time and produce clean NMR spectra. However, for picomole samples, both the conventional and fast NMR have noise in the spectra which make it more difficult for researchers to analyze the spectra. So to overcome the second obstacle, systems and methods according to present principles employ a deep learning system such as a CNN based system that cuts the time for accurate structural determination from weeks or months to minutes or few seconds even in presence of 2D NMR spectral noise. Besides time saving, this technique also greatly reduces the educational access level required. Previously, for structural determination, it was required to have a PhD student trained for 3 years to do the job. Systems and methods according to present principles mimic how seasoned professors (thirty plus years of research) determine structures, and, furthermore, the same capitalize on the wealth of 50 years of NMR based structural determination development in form a database of 2D NMR. In this regard, even undergraduates can perform accurate structural determination.

Thus, systems and methods according to present principles may be employed to effectively and significantly increase the speed and efficiency at which the combination of machines operates, i.e., the 2-D NMR and the deep learning machine. In this way, for each of these machines, the number of cycles required for calculations is reduced and in some cases so is battery power, leading to each machine individually operating better technologically. In addition, the combination of the two machines and subsequent efficiencies leads to a synergistic significant increase in the efficiency of the technological combination. In addition, to the extent either includes a general-purpose computer to perform certain computing functions, the programming of the general-purpose computer leads to the transformation of the general-purpose computer, i.e., hardware, into a special-purpose machine, programmed specifically for that purpose, i.e., 2-D NMR functionality and deep learning machine functionality. In other words, the hardware is transformed into a special-purpose machine. Finally, the results from the combination machine, e.g., the results from the deep learning part of the machine, may be directly transmitted to drug synthesis tools to enable the fabrication of specialty or designer compounds, and this transmission can occur even without the intervention of an operator. The result of systems and methods according to present principles can be directly transmitted to compound or drug fabrication tools, to control and entirely drive the operation of the same, these tools causing a transformation of material from one form, e.g., constituent elements, to another form, e.g., a designed compound.

Finally, the same may serve as a sort of “Facebook” of molecules, where the clustering map is similar to a user's “friends” or “connections”. In this metaphor, the HSQC spectra may be thought of as photos in a family album. Bioactivity or collection data may be analogized to an “about” section, “education”, or “hobbies”. Molecules can be tagged like friends are tagged in a picture. Unknown molecules can be “invited” to join the community. Unknown compound recognition may be analogized to “finding friends”. Molecular structures may evolve to have better bioactivity, e.g., an anticancer function. This can be recorded in their timelines, which will give researchers better ideas to design drugs. Multiple constituents in the same drug product may be analogized to marriage, in the same may be used if the functions of the constituents are known, e.g., to cure a disease. In some cases, small molecules may be considered to “date” a few proteins before they bind to one. A virtual drug screening may be analogized to an “event”, and similar chemical structures may be considered to be interested in going to the same event. An event may also be open to molecules of novel structures. Various view modes may be employed, including a “Google map view”, as well as an “NSA view” that provides additional data.

The system and method may be fully implemented in any number of computing devices. Typically, instructions are laid out on computer readable media, generally non-transitory, and these instructions are sufficient to allow a processor in the computing device to implement the method of the invention. The computer readable medium may be a hard drive or solid state storage having instructions that, when run, are loaded into random access memory. Inputs to the application, e.g., from the plurality of users or from any one user, may be by any number of appropriate computer input devices. For example, users may employ a keyboard, mouse, touchscreen, joystick, trackpad, other pointing device, or any other such computer input device to input data relevant to the calculations. Data may also be input by way of an inserted memory chip, hard drive, flash drives, flash memory, optical media, magnetic media, or any other type of file—storing medium. The outputs may be delivered to a user by way of a video graphics card or integrated graphics chipset coupled to a display that maybe seen by a user. Alternatively, a printer may be employed to output hard copies of the results. Given this teaching, any number of other tangible outputs will also be understood to be contemplated by the invention. For example, outputs may be stored on a memory chip, hard drive, flash drives, flash memory, optical media, magnetic media, or any other type of output. It should also be noted that the invention may be implemented on any number of different types of computing devices, e.g., personal computers, laptop computers, notebook computers, net book computers, handheld computers, personal digital assistants, mobile phones, smart phones, tablet computers, and also on devices specifically designed for these purpose. In one implementation, a user of a smart phone or wi-fi—device downloads a copy of the application to their device from a server using a wireless Internet connection. Such a networked system may provide a suitable computing environment for an implementation in which a plurality of users provide separate inputs to the system and method. In the below system where drug discovery techniques are contemplated, the plural inputs may allow plural users to input relevant data at the same time.

The above description illustrates various exemplary implementations and embodiments of the systems and methods according to present principles. The invention is not limited to such examples. The scope of the invention is to be limited only by the claims appended hereto, and equivalents thereof.

1. A method of determining data about natural products, comprising:
a. performing a 2D NMR technique on an unknown sample; and
b. performing a deep learning method on the results of the NMR technique.

2. The method of claim 1, wherein the 2D NMR technique is a fast NMR technique that screens for suitable fast and NMR pulse sequences at nanomole/picomole sample scales.

3. The method of claim 1, wherein the 2D NMR technique employs nonuniform sampling or sparse sampling.

4. The method of claim 1, wherein the deep learning method employs a convolutional neural network.

5. The method of claim 4, wherein the convolutional neural network is configured to perform dereplication of the sample.

6. The method of claim 4, further comprising the step of training the convolutional neural network.

7. The method of claim 6, wherein the training dereplicates known compounds both in filtered crude extracts and after purification.

8. The method of claim 4, further comprising using an energy-based model and/or a Siamese network to correlate unknown compounds or their moieties with known compounds or their moieties.

9. The method of claim 8, wherein a Siamese deep convoluted neural network applies an energy-based model, whereby correlations may be readily performed of unknown compounds or their moieties with known compounds or their moieties, respectively, whereby new leads may be quickly identified without having to perform intermediate and labor-intensive steps of structural and stereochemical determination of known compounds of interest.

10. The method of claim 4, wherein the deep learning method performs a step of detection.

11. The method of claim 10, wherein the step of detection detects known compounds in filtered VLC fractions, detects known pure compounds, or detects if the value of a suggested compound is compatible with a pattern in a certain spectra.

12. The method of claim 4, wherein the deep learning method performs a step of ranking.

13. The method of claim 12, wherein the step of ranking determines if a subject sample is more compatible with a first spectrum or with a second spectrum.

14. The method of claim 4, wherein the deep learning method performs a step of analyzing.

15. The method of claim 14, wherein the step of analyzing determines if an HSQC pattern of a first moiety in a first spectrum appeared in the HSQC of a known category of compounds, while the pattern of a second moiety in the first spectrum was previously solved in a prior analysis.

16. The method of claim 1, wherein the NMR techniques include a step of data reconstruction with a combined Poisson Gap and Maximum Entropy Method, giving rise to 2D NMR spectra having an improved signal to noise ratio.

17. A non-transitory computer readable medium, comprising instructions for causing a computing environment to perform the method of claim 1.
